(19) United States
(12) Patent Application Publication
(10) Pub. No.: US 2024/0166687 A1
(43) Pub. Date: May 23, 2024

(54) REGULATORY T CELL-INDUCING PEPTIDE AND COMPOSITION INCLUDING SAME FOR PREVENTING OR TREATING AUTOIMMUNE DISEASE

(71) Applicant: IUCF-HYU (INDUSTRY-UNIVERSITY COOPERATION FOUNDATION HANYANG UNIVERSITY), Seoul (KR)

(72) Inventors: Je-Min CHOI, Seoul (KR); Gilran KIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/283,112

(22) PCT Filed: Mar. 31, 2022
(86) PCT No.: PCT/KR2022/004634

§ 371 Date: Sep. 20, 2023

(30) Foreign Application Priority Data
Mar. 31, 2021 (KR) 10-2021-0041584
Sep. 16, 2021 (KR) 10-2021-0124034
Mar. 31, 2022 (KR) 10-2022-0040191

(51) Int. Cl. C07K 7/08; A61K 38/00; A61P 37/06; C07K 1/107; C07K 7/06; C07K 14/00

(52) U.S. Cl. CPC C07K 7/08; A61P 37/06; C07K 1/1077; C07K 7/06; C07K 14/001; A61K 38/00

(57) ABSTRACT

The present disclosure relates to a composition for preventing or treating autoimmune disease, which is based on a CTLA4-derived, regulatory T cell-inducing peptide. The composition has an excellent effect of inhibiting differentiation into cytotoxic Tₕ17 cells that produce and secrete inflammatory cytokines and increasing differentiation into regulatory T cells (T_{reg}) and, thus, can be advantageously utilized as a pharmaceutical composition that can prevent or treat autoimmune disease caused by regulatory anomalies of various immune responses.

### TECHNICAL FIELD

The present disclosure relates to a composition for preventing or treating autoimmune disease, which is based on a regulatory T cell-inducing peptide.

### BACKGROUND ART

Autoimmune disease is an anomaly of the human body's immune system. Whereas a normal immune response protects the body from external antigens such as pathogens, in autoimmune response, the immune system recognizes its own cells as external antigens and attacks its own organs or tissues, causing various diseases in the body.

Rheumatoid arthritis and lupus are known as representative autoimmune diseases and, in addition to systemic scleroderma, atopic dermatitis, multiple sclerosis, Graves' disease and Hashimoto's disease, Crohn's disease, Behçet's disease, Sjögren syndrome, Guillain-Barré syndrome, etc. known as rare diseases are diagnosed as autoimmune diseases. The US National Institutes of Health reports that 80 autoimmune diseases have been identified and women are more commonly affected than men. Recently, with the publication of a research that ‘Tₕ17 cells and regulatory T cells (T_{reg} cells) of the immune system play an important role in breaking the tolerance of normal human immune response and triggering autoimmune response’, researches on autoimmune disease have expanded and become more active.

Systemic autoimmune disease is a disease in which the immune system is abnormally activated and attacks various organs of the body. It is characterized in that antibodies recognize various tissues and organs as self-antigens and destroy one's own cells. Systemic autoimmune disease causes inflammation throughout the body especially by causing abnormalities in connective tissues, such as rheumatoid arthritis, systemic lupus erythematosus, Sjögren syndrome, mixed connective tissue disease, etc.

Localized autoimmune disease (or organ-specific autoimmune disease) refers to a disease that can occur as the body's immune system attacks a specific organ and damages normal cells. Organ-specific autoimmune disease is caused by an abnormal immune response against organ-specific antigens and can occur in almost all organs of the body. Organ damage caused by autoimmune disease can be caused by T cells or by autoantibodies.

Multiple sclerosis is a chronic neuroimmunologic disease affecting the central nervous system including the brain, spinal cord and optic nerve. Although the mechanism of this disease is not known precisely, it is a demyelinating disease (in which the myelin sheath surrounding the axons of neurons is lost) with various symptoms occurring as the myelin sheath surrounding the nerve is damaged due to autoimmune disease caused by T cells and neurotransmission from the brain to various body parts is disturbed.

Multiple sclerosis may be classified into relapsing-remitting multiple sclerosis (RRMS), secondary progressive multiple sclerosis (SPMS), primary progressive multiple sclerosis (PPMS) and progressive relapsing multiple sclerosis (PRMS).

Crohn's disease is a chronic inflammatory bowel disease that may affect any segment of the gastrointestinal tract, from the mouth to the anus. Unlike ulcerative colitis, inflammation invades all layers of the intestine and the distribution of pathological changes is not continuous but often occurs sparsely. The disease occurs most often in the junction between the large intestine and the small intestine, followed by the large intestine, distal ileum, small intestine, etc.

The two autoimmune diseases of thyroiditis are Hashimoto's disease and Graves' disease.

Psoriasis is a chronic, non-infectious skin disease that affects keratinocytes which account for the most part of the epidermis.

The current technology for treating autoimmune disease is an approach for alleviating symptoms or slowing down the progression thereof using anti-inflammation agents, steroids, etc. and mostly relies on empirical effects, and there is no fundamental technology that eliminates the cause of the disease. In cases of severe autoimmune disease, a method of weakening the immune function by administering an immunosuppressant is sometimes selected. But, since this treatment technology has the side effect of bringing fatal results to infectious diseases because the immune function of protecting against bacteria, etc. in healthy adults is also suppressed, it cannot be used for a long period of time.

It is known that the number and function of regulatory T cells are reduced in autoimmune disease, and the induction of regulatory T cells in vivo has been considered as a major treatment strategy.

### DISCLOSURE

### Technical Problem

The present disclosure is directed to providing a regulatory T cell-inducing peptide.

The present disclosure is also directed to providing a pharmaceutical composition for treating and preventing autoimmune disease, which contains a regulatory T cell-inducing peptide as an active ingredient.

### Technical Solution

The present disclosure provides a regulatory T cell-inducing peptide represented by the following sequence.

[Lys-Xaa1-Leu-Xaa2-Xaa3-Arg-Xaa4]ₙ

In the above sequence,
each of Xaa1, Xaa2, Xaa3 and Xaa4 is independently an amino acid selected from alanine (A), valine (V), isoleucine (I), leucine (L), methionine (M), phenylalanine (F), tyrosine (Y), tryptophan (W), lysine (K), arginine (R), histidine (H), serine (S), threonine (T), asparagine (N), glutamine (Q), aspartate (D), glutamate (E), cysteine (C), glycine (G) and proline (P),
n is an integer 2 or 3, and
one or more amino acid residue selected from the above sequence is an L- or D-amino acid residue.

In the sequence, Xaa1 may be a hydrophobic amino acid selected from alanine (A), valine (V), isoleucine (I), leucine (L), methionine (M), phenylalanine (F), tyrosine (Y) and tryptophan (W).

In the sequence, each of Xaa2 and Xaa3 may be independently selected from: a basic amino acid consisting of lysine (K), arginine (R) and histidine (H); and alanine (A).

In the sequence, Xaa4 may be selected from: a polar amino acid consisting of serine (S), threonine (T), asparagine (N) and glutamine (Q); and alanine (A).

In the sequence, Xaa1 may be methionine (M).

In the sequence, Xaa2 and Xaa3 may be lysine (K).

In the sequence, Xaa4 may be serine (S).

In the sequence, one or more amino acid residue selected from the first, second, sixth and seventh positions may be a D-amino acid.

In the sequence, the amino acid residues at the sixth and seventh positions may be D-amino acids.

The regulatory T cell-inducing peptide may further include a linker.

The linker may be selected from a group consisting of (GGGGS)ₘ (1≤m≤4), (GG)ₘ (1≤m≤4), (GSSGGS)ₘ (1≤m≤4), (EAAAK)ₘ (1≤m≤4), PAPAP, (AP)ₘ (1≤m≤4), A(EAAAK)ₘ (1≤m≤4), (RR)ₘ (1≤m≤4) and GFLG.

A fatty acid may be additionally bound to the N- or C-terminal of the regulatory T cell-inducing peptide.

The fatty acid may be bound to the N-terminal of the regulatory T cell-inducing peptide via an amide bond.

The fatty acid may be a C₁₀₋₂₀ saturated or unsaturated fatty acid.

The fatty acid may be one or more selected from a group consisting of myristic acid, stearic acid, linoleic acid, palmitic acid, oleic acid and lauric acid.

A cell-penetrating peptide (CPP) may be additionally bound to the N- or C-terminal of the regulatory T cell-inducing peptide.

The cell-penetrating peptide may be one or more selected from a group consisting of dNP2 represented by SEQ ID NO 18, AP represented by SEQ ID NO 19, TAT (HIV 1 trans-activating protein), a polyarginine polypeptide having 6 to 8 arginines, a polylysine polypeptide having 7 to 11 lysines and iRGD (internalizing RGD).

The regulatory T cell-inducing peptide may be any one selected from a group consisting of SEQ ID NOS 1-6, SEQ ID NO 11, SEQ ID NO 12 and SEQ ID NOS 50-54.

The regulatory T cell-inducing peptide with the cell-penetrating peptide bound may be any one selected from a group consisting of SEQ ID NOS 7-10, SEQ ID NO 13 and SEQ ID NO 14.

The present disclosure also provides a pharmaceutical composition for preventing or treating autoimmune disease, which contains the regulatory T cell-inducing peptide as an active ingredient.

The autoimmune disease may be one or more selected from a group consisting of lupus (systemic lupus erythematosus), rheumatoid arthritis, Crohn's disease, ulcerative colitis, systemic scleroderma (progressive systemic sclerosis), atopic dermatitis, psoriasis, pemphigus, asthma, aphthous stomatitis, chronic thyroiditis, inflammatory enteritis, multiple sclerosis, mixed connective tissue disease, autoimmune hemolytic anemia, Behçet's disease, autoimmune encephalomyelitis, myasthenia gravis, autoimmune thyroiditis, polyarteritis nodosa, ankylosing spondylitis, fibromyalgia syndrome, Sjögren syndrome, autoimmune uveitis, chronic inflammatory demyelinating polyneuropathy and temporal arteritis.

The composition may inhibit the activity of Tₕ17 and activate T_{reg}.

The present disclosure also provides a use of a pharmaceutical composition containing the regulatory T cell-inducing peptide as an active ingredient for preparation of a drug for preventing or treating autoimmune disease.

The present disclosure also provides a method for treating autoimmune disease, which includes administering a pharmaceutical composition containing the regulatory T cell-inducing peptide as an active ingredient to a patient with autoimmune disease.

Hereinafter, the present disclosure will be described with reference to the accompanying drawings. However, the present disclosure may be embodied in many different forms and thus is not limited to the exemplary embodiments described herein. In the drawings, the parts irrelevant to the description have been omitted to clearly explain the present disclosure and similar reference numerals have been used to indicate similar parts throughout the specification.

Throughout the specification, when a part is said to be “connected (contacted or combined)” with another part, this includes not only the case where it is “connected directly” but also the case where it is “connected indirectly” with another member in between. In addition, when a part is said to “include” a certain component, this means that it may further include other components rather than excluding other components, unless the context indicates otherwise.

The terms used in the present specification are used only to describe specific exemplary embodiments and are not intended to limit the present disclosure. Singular expressions include plural expressions unless the context explicitly dictates otherwise. In the present specification, the terms such as “include”, “have”, etc. are intended to indicate the existence of the features, numbers, steps, operations, components, parts or combinations thereof described in the specification and it should be understood that the existence or addition of one or more other features, numbers, steps, operations, components, parts or combinations thereof is not precluded.

In an aspect, the present disclosure relates to a regulatory T cell-inducing peptide represented by the following sequence.

[Lys-Xaa1-Leu-Xaa2-Xaa3-Arg-Xaa4]ₙ

In the sequence, each of Xaa1, Xaa2, Xaa3 and Xaa4 is independently an amino acid residue selected from alanine (A), valine (V), isoleucine (I), leucine (L), methionine (M), phenylalanine (F), tyrosine (Y), tryptophan (W), lysine (K), arginine (R), histidine (H), serine (S), threonine (T), asparagine (N), glutamine (Q), aspartate (D), glutamate (E), cysteine (C), glycine (G) and proline (P), n is an integer 2 or 3, and one or more amino acid residue selected from the sequence is an L- or D-amino acid residue.

In the present disclosure, the term ‘peptide’ refers to a polymer in the form of a chain formed as 2 to 1000 amino acid residues are linked by peptide bonds and it is interchangeable with ‘polypeptide’.

In the present disclosure, the peptide preferably has the amino acid sequence described above, although not being limited thereto. In a specific exemplary embodiment of the present disclosure, the peptide contains the above amino acid at a ratio of 50% or higher, specifically 60% or higher, more specifically 70% or higher, more specifically 80% or higher, more specifically 90% or higher, most specifically 100%.

In the present disclosure, the term ‘polynucleotide’ refers to a polymer compound wherein nucleotides, i.e., chemical monomers consisting of three elements, a base, a sugar and a phosphate, are connected in a chain form via a plurality of phosphate ester bonds.

The regulatory T cell-inducing peptide of the present disclosure may be a peptide derived from the CTLA4 protein (cytotoxic T-lymphocyte antigen-4), consisting of 14 to 31 amino acids, and one or more of the amino acid residue may be an L- or D-amino acid residue.

Specifically, in the sequence, Xaa1 may be a hydrophobic amino acid selected from alanine (A), valine (V), isoleucine (I), leucine (L), methionine (M), phenylalanine (F), tyrosine (Y) and tryptophan (W). Specifically, in the sequence, Xaa1 may be methionine (M).

Specifically, in the sequence, each of Xaa2 and Xaa3 may independently be a basic amino acid selected from lysine (K), arginine (R) and histidine (H). Specifically, in the sequence, Xaa2 and Xaa3 may be the same amino acid. More specifically, Xaa2 and Xaa3 may be lysine (K).

In addition, in the sequence, Xaa4 may be a polar amino acid selected from serine (S), threonine (T), asparagine (N) and glutamine (Q). Specifically, Xaa4 may be serine (S).

Most specifically, in the sequence, Xaa1 may be methionine (M), Xaa2 and Xaa3 may be lysine (K), Xaa4 may be serine (S). The regulatory T cell-inducing peptide may be represented by represented by General Formula 2. It is distinguished from the ‘sequence’ described in claim 1 and it not used interchangeably with the term ‘sequence’ in the present disclosure. Without the mentioning of General Formula 2, the ‘sequence’ refers to ‘[Lys-Xaa1-Leu-Xaa2-Xaa3-Arg-Xaa4]ₙ’ described in claim 1 in the present disclosure.

[Lys-Met-Leu-Lys-Lys-Arg-Ser]ₙ (2≤n≤3) [General Formula 2]

Specifically, in the sequence, one or more amino acid residue selected from the first, second, sixth and seventh positions may be a D-amino acid. Most specifically, in the sequence, one or more amino acid residue selected from the lysine residue at the first position, the methionine residue at the second position, the arginine residue at the sixth position and the serine residue at the seventh position may be a D-amino acid.

In the sequence, the amino acid residues at the sixth position and the seventh position may be D-amino acids. Most specifically, in the sequence, the arginine residue at the sixth position and the serine residue at the seventh position may be D-amino acids.

But, in the sequence, if n is an integer 3, the lysine residue at the first position, the methionine residue at the second position, the arginine residue at the sixth position, the arginine residue at the thirteenth position, the arginine residue at the twenty-first position, the serine residue at the seventh position, the serine residue at the fourteenth position and the serine residue at the twenty-first position may be D-amino acids.

The regulatory T cell-inducing peptide according to the present disclosure has superior effect of inducing and activating regulatory T cells even when a cell-penetrating peptide is not bound, and has the effect of inducing differentiation into regulatory T cells (T_{reg} cells) and inhibiting differentiation into Tₕ17 cells. Therefore, unlike CTLA4, it can be synthesized easily, has improved stability, and can maintain superior therapeutic effect and achieve excellent pharmacological properties in vivo. In addition, since modification at the N- and/or C-terminal of the peptide to achieve the desired formulation or necessary properties is easy, it is remarkably superior to the CTLA4 with a long chain in terms of utility.

In addition, the D-amino acid residue is advantageous over the L-amino acid residue in terms of stability and the ability of regulating immune function when symptoms are induced by T_{reg} cells, i.e., excessive immune response.

In the present disclosure, the peptide may be obtained by various methods widely known in the art. Specifically, it can be prepared using genetic recombination and protein expression systems or can be synthesized in vitro through chemical synthesis such as peptide synthesis, cell-free protein synthesis, etc.

CTLA4 is a receptor existing in cytotoxic T cells. It is known to block activation signals of T cells and mediate negative signal transduction by binding to B7 on antigen-presenting cells. It is also known to play an important role in the immunosuppressive mechanism of regulatory T cells. Although the function of the CTLA4 protein or CTLA4 cytoplasmic domain has been identified, there is a limitation in that it has to be delivered into cells through binding to a cell-penetrating peptide to exert its effect. Therefore, for more fundamental treatment of autoimmune disease, a new therapeutic agent is necessary.

The regulatory T cell-inducing peptide may further include a linker.

In the present disclosure, the ‘linker’ may be provided in the middle of the regulatory T cell-inducing peptide and may provide flexibility. The linker may be located anywhere in the regulatory T cell-inducing peptide, but specifically between the repeating units of the sequence represented by [Lys-Met-Leu-Lys-Lys-Arg-Ser]. That is to say, it may be located at the site where the serine residue and the lysine residue are linked. The linker is generally composed of amino acids, but its type and length are not specially limited. In an exemplary embodiment of the present disclosure, it may be one or more selected from a group consisting of (GGGGS)ₘ (1≤m≤4), (GG)ₘ (1≤m≤4), (GSSGGS)ₘ (1≤m≤4), (EAAAK)ₘ (1≤m≤4), PAPAP, (AP)ₘ (1≤m≤4), A(EAAAK)ₘ (1≤m≤4), (RR)ₘ (1≤m≤4) and GFLG. In (GGGGS)ₘ, four glycines and one serine may be arranged repeatedly. In (GG)ₘ, two glycines may be arranged repeatedly. In (GSSGGS)ₘ, glycine-serine-serine-glycine-glycine-serine may be arranged repeatedly. (EAAAK)ₘ and A(EAAAK)ₘ (1≤m≤4) (wherein E is glutamate, A is alanine, K is lysine, G is glycine, S is serine, and m is generally an integer 1 or larger, specifically an integer from 1 to 4) are alpha-helix linkers having rigid properties. In addition, PAPAP, (AP)ₘ (1≤m≤4), (RR)ₘ (1≤m≤4) and GFLG (wherein P is proline, A is alanine, R is arginine, F is phenylalanine, and L is lysine) may be used.

In the present disclosure, the peptide may include a targeting sequence, a tag, a labeled residue, or an additional amino acid sequence designed for special purpose to increase the half-life or stability of the peptide. In addition, the peptide of the present disclosure may be linked to a coupling partner such as an effector, a drug, a prodrug, a toxin, a peptide, a carrier molecule, etc.

A fatty acid may be additionally bound to the N- or C-terminal of the regulatory T cell-inducing peptide of the present disclosure to ensure in-vivo stability.

The fatty acid may be bound to the N-terminal of the regulatory T cell-inducing peptide via an amide bond. Specifically, the carboxyl group of the fatty acid may be bound to the amino group of the peptide by an amide bond.

The fatty acid may be a C₁₀₋₂₀ saturated or unsaturated fatty acid, specifically one or more selected from a group consisting of myristic acid, stearic acid, linoleic acid, palmitic acid, oleic acid and lauric acid, most specifically myristic acid (also abbreviated as ‘Myr’) or palmitic acid (also abbreviated as ‘Pal’).

The regulatory T cell-inducing peptide of the present disclosure can provide high therapeutic effect without loss or deterioration of its original function even when the fatty acid is bound.

In addition, a cell-penetrating peptide (CPP) may be additionally bound to the N- or C-terminal of the regulatory T cell-inducing peptide to enhance cell-penetrating activity.

In the present disclosure, the term ‘cell-penetrating peptide (CPP)’ refers to a peptide that has the ability to deliver a cargo into cells in vitro or in vivo, and can be used interchangeably with ‘cell membrane-penetrating domain’ or ‘cell membrane-penetrating peptide’.

The cell-penetrating peptide may be one or more selected from a group consisting of dNP2 represented by SEQ ID NO 18, AP represented by SEQ ID NO 19, TAT (HIV 1 trans-activating protein), polyarginine polypeptide having 6 to 8 arginines, polylysine polypeptide having 7 to 11 lysines and iRGD (internalizing RGD), specifically one or more sequence selected from SEQ ID NO 18, SEQ ID NO 19 and SEQ ID NOS 33-41, more specifically a linker represented by SEQ ID NO 18 or SEQ ID NO 19.

The regulatory T cell-inducing peptide and the fatty acid or the cell-penetrating peptide may be linked directly or indirectly by further including a linker peptide therebetween. As the linker peptide, amino acids known in the art or peptides composed of combinations thereof may be used without limitation. Specifically, the linker peptide may be one or more selected from a group consisting of (GGGGS)ₘ (1≤m≤4), (GG)ₘ (1≤m≤4), (GSSGGS)ₘ (1≤m≤4), (EAAAK)ₘ (1≤m≤4), PAPAP, (AP)ₘ (1≤m≤4), A(EAAAK)ₘ (1≤m≤4), (RR)ₘ (1≤m≤4) and GFLG.

In another specific exemplary embodiment of the present disclosure, the regulatory T cell-inducing peptide may be linked to the fatty acid, the cell-penetrating peptide or both the fatty acid and the cell-penetrating peptide. Specifically, the fatty acid may be bound after the cell-penetrating peptide is bound to the regulatory T cell-inducing peptide, although there is no limitation in the sequence.

In the present disclosure, the function of each motif of CTLA4 was identified and it was confirmed that the K motif of the CTLA4 protein plays a major role. However, only with the K motif, the effect of alleviating symptoms could not be achieved in vivo because the effect of inducing regulatory T cells cannot be obtained and the effect of inhibiting Tₕ17 cell activity is insignificant. In the present disclosure, it was confirmed that a regulatory T cell-inducing peptide with two or three K motifs linked has the effect of inducing regulatory T cells and inhibiting Tₕ17 cell activity and also exhibits superior effect of alleviating symptoms in vivo.

Specifically, the regulatory T cell-inducing peptide of the present disclosure may be selected from a group consisting of SEQ ID NOS 1-6, SEQ ID NO 11, SEQ ID NO 12 and SEQ ID NOS 50-54. More specifically, the regulatory T cell-inducing peptide may be selected from a group consisting of SEQ ID NOS 1-6, SEQ ID NO 11 and SEQ ID NO 12.

The regulatory T cell-inducing peptide of the present disclosure with the fatty acid or cell-penetrating peptide bound may be any one selected from a group consisting of SEQ ID NOS 7-10, SEQ ID NO 13 and SEQ ID NO 14.

In the present disclosure, it was confirmed that the K motif of the CTLA4 protein the function of which is not specifically known is involved in the inhibition of immune response but therapeutic effect cannot be achieved in vivo only with the K motif. Through numerous experiments, it was confirmed that the regulatory T cell-inducing peptide according to the present disclosure exhibits the effect of preventing, treating or alleviating autoimmune disease.

Unlike the CTLA4 protein, the regulatory T cell-inducing peptide according to the present disclosure not only exhibits superior effect of preventing or treating autoimmune disease even when a carrier for intracellular penetration such as a cell-penetrating peptide, etc. is not bound and effectively inhibits immune response by targeting Tₕ17 and T_{reg} cells. Therefore, it can be used as a therapeutic agent for autoimmune disease.

It was confirmed that the regulatory T cell-inducing peptide according to the present disclosure remarkably alleviates the symptoms of autoimmune disease in vivo, significantly alleviates only the symptoms without affecting survival rate at all in an animal model of multiple sclerosis even when it has D-amino acids for improvement of stability, effectively inhibits the production of IL-17 by inhibiting Tₕ17 activation, increases the activation of T_{reg} cells which inhibit immune response, and negatively regulates the production of IFN-gamma such as Tₕ1.

Accordingly, the regulatory T cell-inducing peptide of the present disclosure may be usefully used as a drug for preventing or treating autoimmune disease.

The present disclosure relates to a pharmaceutical composition for preventing or treating autoimmune disease, which contains the regulatory T cell-inducing peptide as an active ingredient.

The pharmaceutical composition of the present disclosure may further contain a pharmaceutically acceptable carrier. The pharmaceutically acceptable carrier includes lactose, dextrose, sucrose, sorbitol, mannitol, starch, acacia gum, calcium phosphate, alginate, gelatin, calcium silicate, microcrystalline cellulose, polyvinylpyrrolidone, water, syrup, methyl cellulose, methyl hydroxybenzoate, propyl hydroxybenzoate, talc, magnesium stearate, mineral oil, etc., although not being limited thereto. The pharmaceutical composition of the present disclosure may further contain, in addition to the above ingredients, a lubricant, a wetting agent, a sweetener, a flavorant, an emulsifier, a suspending agent, a preservative, etc. Suitable pharmaceutically acceptable carriers and formulations are described in detail in Remington's Pharmaceutical Sciences (19th ed., 1995).

In the present disclosure, the term “prevention” refers to any action of suppressing or delaying autoimmune disease by administering a composition containing the regulatory T cell-inducing peptide of the present disclosure as an active ingredient.

In the present disclosure, the term “treatment” refers to any action of alleviating or favorably changing autoimmune disease by administering a composition containing the regulatory T cell-inducing peptide of the present disclosure as an active ingredient.

In the present disclosure, the ‘autoimmune disease’ refers to a disease that occurs as immune cells, which should protect the body from foreign organisms such as bacteria, viruses, etc., recognize one's own cells as external antigens and attack them due to anomaly.

The autoimmune disease may be one or more selected from a group consisting of lupus (systemic lupus erythematosus), rheumatoid arthritis, Crohn's disease, ulcerative colitis, systemic scleroderma (progressive systemic sclerosis), atopic dermatitis, psoriasis, pemphigus, asthma, aphthous stomatitis, chronic thyroiditis, inflammatory enteritis, multiple sclerosis, mixed connective tissue disease, autoimmune hemolytic anemia, Behçet's disease, autoimmune encephalomyelitis, myasthenia gravis, autoimmune thyroiditis, polyarteritis nodosa, ankylosing spondylitis, fibromyalgia syndrome, Sjögren syndrome, autoimmune uveitis, chronic inflammatory demyelinating polyneuropathy and temporal arteritis.

Autoimmune disease is mostly induced by an imbalance of Tₕ1 and Tₕ2 immune cells due to abnormally increased activity of the Tₕ1 cells. Regulatory T cells (T_{reg}) regulate inflammatory responses by suppressing the function of abnormally activated immune cells. In addition, Tₕ17 cells are produced during differentiation. Tₕ17 cells are formed through a process similar to that of T_{reg} cells during the differentiation of undifferentiated T cells. The differentiated Tₕ17 cells secrete IL-17, which is known to be involved in inflammatory responses seen in autoimmune disease and accelerate the progression of the disease by maximizing the signals of the inflammatory responses. In this regard, most therapeutic agents for autoimmune disease are being developed by targeting the inhibition of the activity of Tₕ1 and Tₕ17 cells, but drugs targeting T_{reg} cells have not been approved yet. Conventional immunosuppressants used as therapeutic agents for autoimmune disease have side effects such as toxicity, infection, lymphoma, diabetes, tremor, headache, diarrhea, hypertension, nausea, renal dysfunction, etc. Therefore, there is a need to develop a therapeutic agent which has no side effect, is inexpensive and has superior therapeutic effect.

The inventors of the present disclosure have made efforts to develop a substance that can overcome the limitations of the existing therapeutic agents for autoimmune disease and replace them. As a result, they have found out that the K motif among the motifs present in CTLA4 has an effect but cannot exert a sufficient effect alone. They have found out that autoimmune disease can be treated very effectively when two or three of them are linked, and have completed the present disclosure.

That is to say, since the composition according to the present disclosure, which contains the regulatory T cell-inducing peptide as a single active ingredient, can inhibit the activity of Tₕ17 cells and induce the activation of T_{reg} cells at the same time, it can effectively prevent or treat autoimmune disease by suppressing immune response of autoimmune disease.

In addition, the pharmaceutical composition for preventing and treating autoimmune disease of the present disclosure may contain a pharmaceutically acceptable carrier.

The pharmaceutically acceptable carrier may include, for example, a carrier for oral administration or a carrier for parenteral administration. The carrier for oral administration may include lactose, starch, a cellulose derivative, magnesium stearate, stearic acid, etc. And, the carrier for parenteral administration may include water, a suitable oil, saline, water-soluble glucose, glycol, etc. In addition, a stabilizer and a preservative may be further included. Suitable stabilizers include an antioxidant such as sodium bisulfite, sodium sulfite or ascorbic acid. Suitable preservatives include benzalkonium chloride, methyl- or propylparaben and chlorobutanol. For other pharmaceutically acceptable carriers, reference may be made to those described in the following literature (Remington's Pharmaceutical Sciences, 19th ed., Mack Publishing Company, Easton, PA, 1995).

The pharmaceutical composition of the present disclosure may be administered to mammals including human by any method. For example, it may be administered orally or parenterally. The parenteral administration may be made by intravenous, intramuscular, intraarterial, intramedullary, intrathecal, intracardiac, transdermal, subcutaneous, intraperitoneal, intranasal, enteral, topical, sublingual or rectal administration, although not being limited thereto.

The pharmaceutical composition of the present disclosure may be prepared into a formulation for oral or parenteral administration depending on the administration route as described above. The formulation may be prepared using one or more buffer (e.g., saline or PBS), antioxidant, bacteriostat, chelating agent (e.g., EDTA or glutathione), filler, extender, binder, adjuvant (e.g., aluminum hydroxide), suspending agent, thickener, wetting agent, disintegrant, surfactant, diluent or excipient.

Solid formulations for oral administration include a tablet, a pill, a powder, a granule, a liquid, a gel, a syrup, a slurry, a suspension, a capsule, etc. These solid formulations may be prepared by mixing the pharmaceutical composition of the present disclosure with at least one excipient, e.g., starch (corn starch, wheat starch, rich starch, potato starch, etc.), calcium carbonate, sucrose, lactose, dextrose, sorbitol, mannitol, xylitol, erythritol, maltitol, cellulose, methyl cellulose, sodium carboxymethyl cellulose, hydroxypropylmethyl cellulose, gelatin, etc. For example, a tablet or a sugar-coated tablet may be obtained by mixing the active ingredient with a solid excipient, pulverizing the mixture, adding a suitable adjuvant and then processing the mixture into a granule.

In addition to a simple excipient, a lubricant such as magnesium stearate and talc is also used. Liquid formulations for oral administration include a suspension, a liquid formulation for internal use, an emulsion, a syrup, etc. In addition to commonly used simple diluents such as water or liquid paraffin, various excipients such as a wetting agent, a sweetener, an aromatic, a preservative, etc. may be included.

In addition, disintegrants such as crosslinked polyvinylpyrrolidone, agar, alginic acid, sodium alginate, etc. may be added if necessary, and an anticoagulant, a lubricant, a wetting agent, a flavorant, an emulsifier, an antiseptic, etc. may be further included.

For parenteral administration, an injection, a formulation for transdermal administration or a nasal inhalant may be prepared according to methods known in the art using the pharmaceutical composition of the present disclosure together with a suitable carrier for parenteral administration. The injection should be sterilized and protected against contamination by microorganisms such as bacteria or fungi. Carriers suitable for the injection include solvents or dispersion media including water, ethanol, a polyol (e.g., glycerol, propylene glycol, liquid polyethylene glycol, etc.), mixtures thereof and/or vegetable oil, although not being limited thereto. More specifically, isotonic solutions such as Hank's solution, Ringer's solution, triethanolamine-containing PBS (phosphate-buffered saline), sterilized water for injection, 10% ethanol, 40% propylene glycol, 5% dextrose solution may be used as suitable carriers. The injection may further contain various antibacterial or antifungal agents such as paraben, chlorobutanol, phenol, sorbic acid, thimerosal, etc. for protection from contamination by microorganisms. In addition, the injection may further contain an isotonic agent such as sugar or sodium chloride in most cases.

The formulation for transdermal administration includes an ointment, a cream, a lotion, a gel, a liquid for external use, a paste, a liniment, an aerosol, etc. The ‘transdermal administration’ refers to the delivery of an effective amount of the active ingredient contained in the pharmaceutical composition through topical application to skin.

For the formulation for administration by inhalation, the active ingredient of the present disclosure may be delivered conveniently in the form of an aerosol spray from a compressed pack or a nebulizer using a suitable propellant, e.g., dichlorofluoromethane, trichlorofluoromethane, dichlorotetrafluoroethane, carbon dioxide or other suitable gas. For a compressed aerosol, a dosage unit may be determined by providing a valve that delivers a metered amount. For example, gelatin capsules and cartridges used in inhalers or insufflators may be formulated to contain a mixture of the active ingredient and lactose or starch. The formulations for parenteral administration are described in the literature known in the art (Remington's Pharmaceutical Science, 15th Edition, 1975. Mack Publishing Company, Easton, Pennsylvania 18042, Chapter 87: Blaug, Seymour).

The pharmaceutical composition for preventing and treating autoimmune disease of the present disclosure, which contains an effective amount of the regulatory T cell-inducing peptide, may specifically provide the effect of preventing and treating autoimmune disease. In the present specification, the ‘effective amount’ refers to an amount that exhibits a greater response than that of a negative control group, specifically an amount sufficient to prevent or treat autoimmune disease. In the pharmaceutical composition of the present disclosure, the amount of the regulatory T cell-inducing peptide may be 0.01-99.99% and the remaining amount may be occupied by a pharmaceutically acceptable carrier. The effective amount of the regulatory T cell-inducing peptide contained in the pharmaceutical composition of the present disclosure will vary depending on the formulation of the composition, etc.

The total effective amount of the pharmaceutical composition of the present disclosure may be administered to a patient as a single dose or by a fractionated treatment protocol in which multiple doses are administered over a long period of time. The content of the active ingredient in the pharmaceutical composition of the present disclosure may vary depending on the severity of the disease. In the case of parenteral administration, the regulatory T cell-inducing peptide may be administered at a content of specifically 0.0001-50 mg, more specifically 0.001-30 mg, per 1 kg of body weight on a daily basis. In the case of oral administration, the regulatory T cell-inducing peptide may be administered in one or several divided doses at a content of specifically 0.0001-100 mg, more specifically 0.001-50 mg, per 1 kg of body weight on a daily basis. However, the effective administration amount of the regulatory T cell-inducing peptide for a patient is determined in consideration of various factors such as the age, body weight, health status and sex of the patient, the severity of the disease, diet, excretion rate, etc. as well as the administration route and number of the pharmaceutical composition. Those having ordinary knowledge in the art will be able to determine an effective administration amount of the regulatory T cell-inducing peptide appropriate for the prevention and treatment of autoimmune disease in consideration of these factors. The formulation, administration route and administration method of the pharmaceutical composition according to the present disclosure are not specially limited as long as the effect of the present disclosure is exhibited.

The pharmaceutical composition for preventing and treating autoimmune disease of the present disclosure may be used alone or in combination with surgery, radiation therapy, hormone therapy, chemotherapy, biological response modifiers or therapeutic agents for autoimmune disease.

As described above, whereas the conventional therapeutic agents for autoimmune disease are not effective or have side effects when used alone, the regulatory T cell-inducing peptide of the present disclosure can effectively regulate immune response by increasing the activity of T_{reg} cells and inhibiting the activity of Tₕ17 cells at the same time. In addition, it can exhibit superior effect even with the short sequence of 14-31 amino acids. Furthermore, because it exhibits high performance, shows few side effects and can be prepared easily, it may be utilized as a new therapeutic agent for preventing or treating autoimmune disease.

The pharmaceutical composition for preventing and treating autoimmune disease of the present disclosure may also be provided as a formulation for external application, which contains the regulatory T cell-inducing peptide as an active ingredient.

When the pharmaceutical composition for preventing and treating autoimmune disease of the present disclosure is used as a formulation for external application to skin, it may further contain other ingredients commonly used in formulations for external application to skin, such as a fatty substance, an organic solvent, a solubilizer, a thickener, a gelling agent, a softener, an antioxidant, a suspending agent, a stabilizer, a foaming agent, an aromatic, a surfactant, water, an ionic emulsifier, a non-ionic emulsifier, a filler, a sequestrant, a chelating agent, a preservative, a vitamin, a blocking agent, a wetting agent, an essential oil, a dye, a pigment, a hydrophilic activator, a lipophilic activator, a lipid vesicle, etc., and adjuvants commonly used in the field of dermatology. In addition, these ingredients may be introduced in amounts commonly used in the field of dermatology.

When the pharmaceutical composition for preventing and treating autoimmune disease of the present disclosure is provided as a formulation for external application to skin, it may be in the form of an ointment, a patch, a gel, a cream, a spray, etc., although not being limited thereto.

The composition of the present disclosure may be added to a feed additive for preventing or alleviating autoimmune disease or a feed composition containing the same.

In the present disclosure, the term “feed additive” includes substances added to feed for various purposes, such as supplementation of nutrients, reduction of body weight, improvement of digestibility of fiber in feed, improvement of milk quality, prevention of reproductive disorder, improvement of conception rate, prevention of heat stress in summer, etc. The feed additive of the present disclosure corresponds to supplementary feed under the Feed Management Act and may further include minerals such as sodium bicarbonate, bentonite, magnesium oxide, complex mineral, etc. trace minerals such as zinc, copper, cobalt, selenium, etc., vitamins such as carotene, vitamins A, D and E, nicotinic acid, vitamin B complex, etc., protective amino acids such as methionine, lysine, etc., protective fatty acids such as fatty acid calcium salts, etc., and probiotics such as lactic acid bacteria, yeast culture, fermented mold, etc.

In the present disclosure, the term “feed” refers to any natural or artificial diet, meal, or ingredients of the meal for eating, ingestion and digestion by animals. A feed containing the composition for preventing or alleviating autoimmune disease according to the present disclosure as an active ingredient can be prepared into various types of feed known in the art, specifically into concentrated feed, roughage and/or special feed, although not being limited thereto.

The concentrated feed includes seeds including grains such as wheat, oats, corn, etc., brans as byproducts obtained after refining grains, including rice bran, wheat bran, barley bran, etc., seedcakes as byproducts obtained after extracting oil from soybean, rapeseed, sesame, linseed, coconut palm, etc., residues remaining after extracting starch from sweet potato, potato, etc., fish meal, fish offal, fish soluble obtained by concentrating fresh liquid obtained from fish, meat meal, blood meal, feather meal, skim milk, dried whey obtained by drying whey which is a liquid remaining after preparation of cheese, skim milk or casein from milk, yeast, chlorella and seaweed, although not being limited thereto.

The roughage includes raw grass feed such as wild grass, grass, green grass, etc., root vegetables such as turnip for feed, beet for feed, etc., silage which is a storage feed obtained by fermenting raw grass, green grass, crop, etc. filled in a silo with lactic acid bacteria, hay obtained by drying wild grass or pasture, crop straw for breeding and legume leaves, although not being limited thereto. The special feed includes mineral feed such as oyster shell, rock salt, etc., urea feed such as urea or diureidisobutane, which is a derivative thereof, and a feed additive or a dietary supplement added in a small amount to supplement ingredients that are likely to be insufficient or to increase the storage property of the feed, although not being limited thereto.

The feed additive for preventing or alleviating autoimmune disease according to the present disclosure can be prepared by adding the regulatory T cell-inducing peptide according to various feed preparation methods known in the art.

The feed additive according to the present disclosure may be applied to any subject in need of the prevention or alleviation of autoimmune disease without limitation. For example, it can be applied to any subject such as cow, horse, pig, goat, sheep, dog, cat, rabbit, birds, fish, etc.

The present disclosure also provides a method for preventing or treating autoimmune disease, which includes a step of administering the pharmaceutical composition described above.

In the method of the present disclosure, the pharmaceutical composition may be administered to any animal such as chicken, pig, monkey, dog, cat, rabbit, guinea pig, rat, mouse, cow, sheep, goat, etc. without limitation. The animal is not specially limited as long as it is a non-human animal.

In the present disclosure, the administration may be made by any means known in the art. For example, the administration may be made directly into the subject intravenously, intramuscularly, intraperitoneally, orally, transdermally, mucosally, intranasally, intratracheally or subcutaneously. The administration may be made either systemically or topically.

In the method of the present disclosure, the composition of the present disclosure may be administered in a therapeutically or prophylactically effective amount. The “therapeutically or prophylactically effective amount” may be determined adequately by those skilled in the art in consideration of the severity of symptoms and the sex, age, body weight, etc. of the subject. The therapeutically or prophylactically effective amount of the regulatory T cell-inducing peptide may be, for example, 0.0001-100 mg per 1 kg of the body weight of the subject.

### Advantageous Effects

The present disclosure relates to a CTLA4-derived peptide of regulatory T cells. A composition containing the CTLA4-derived peptide has an excellent effect of inhibiting differentiation into cytotoxic Tₕ17 cells that produce and secrete inflammatory cytokines and increasing differentiation into regulatory T cells (T_{reg}) and, thus, can be advantageously utilized as a pharmaceutical composition that can prevent or treat autoimmune disease caused by regulatory anomalies of various immune responses.

The regulatory T cell-inducing peptide of the present disclosure can effectively suppress and alleviate the severity of autoimmune disease in vivo, and has superior effect of treating or preventing autoimmune disease by inhibiting the activity of Tₕ17 cells and activating T_{reg} cells.

### BRIEF DESCRIPTION OF DRAWINGS

FIG. 1 shows the structure of dNP2-ctCTLA-4 and dNP2-ctCTLA-4 variants (KA, PA and YF).

FIG. 2 shows a result of flow cytometric analysis of IL-17A and foxp3 for five groups (Th17, WT, KA, PA and YF) treated to Tₕ17 cells differentiated from splenocytes isolated from mouse.

FIG. 3 shows a result of flow cytometric analysis of CD4⁺IL-17A⁺ T cells and CD4⁺foxp3⁺ T cells for five groups (Th17, WT, KA, PA and YF) prepared using dNP2-ctCTLA-4 and dNP2-ctCTLA-4 variants (KA, PA and YF).

FIG. 4 shows a result of evaluating clinical symptoms of a multiple sclerosis animal model for five groups (PBS, WT, KA, PA and YF) prepared using dNP2-ctCTLA-4 and dNP2-ctCTLA-4 variants (KA, PA and YF).

FIG. 5 shows a result of analyzing the number of immune cells (Tₕ1, Tₕ17 and T_{reg}) for five groups (PBS, WT, KA, PA and YF) prepared using dNP2-ctCTLA-4 and dNP2-ctCTLA-4 variants (KA, PA and YF).

FIG. 6 shows a result of histological analysis of the spinal cord of a multiple sclerosis animal model to which five groups (PBS, WT, KA, PA and YF) prepared using dNP2-ctCTLA-4 and dNP2-ctCTLA-4 variants (KA, PA and YF) were administered.

FIG. 7 shows the structure of deleted variants of dNP2-ctCTLA-4, dNP2 and dNP2-ctCTLA-4 (KSYPQ, KSYP, KSY, KS and K).

FIG. 8 shows a result of flow cytometric analysis of IL-17A and foxp3 for eight groups (Tₕ17, dNP2, dNP2-ctCTLA4, KSYPQ, KSYP, KSY, KS and K) treated to Tₕ17 cells differentiated from splenocytes isolated from mouse.

FIG. 9 shows a result of flow cytometric analysis of CD4⁺IL-17A⁺ T cells and CD4⁺foxp3⁺ T cells for eight groups (Tₕ17, dNP2, dNP2-ctCTLA4, KSYPQ, KSYP, KSY, KS and K).

FIG. 10 shows a result of evaluating clinical symptoms of a multiple sclerosis animal model for five groups (PBS, dNP2-ctCTLA-4, TAT-ctCTLA-4, KSY and K).

FIG. 11 shows a result of analyzing the number of immune cells (Tₕ1, Tₕ17 and T_{reg}) for five groups (PBS, dNP2-ctCTLA-4, TAT-ctCTLA-4, KSY and K).

FIG. 12 shows the structure of dNP2-ctCTLA-4 (WT), dNP2, dNP2-ctCTLA-4 variants (K, S, Y1, P and Y2).

FIG. 13 shows a result of flow cytometric analysis of IL-17A and foxp3 for eight groups (Tₕ17, dNP2, dNP2-ctCTLA-4 (WT), K, S, Y1, P and Y2) treated to Tₕ17 cells differentiated from splenocytes isolated from mouse.

FIG. 14 shows a result of flow cytometric analysis of CD4⁺IL-17A⁺ T cells and CD4⁺foxp3⁺ T cells for eight groups (Tₕ17, dNP2, dNP2-ctCTLA-4, K, S, Y1, P and Y2). F means Y2.

FIG. 15 shows the structure of dNP2-ctCTLA-4 (WT), K1, K2 (G4S), K3 (G4S), K3 (GG) and K3 (no linker).

FIG. 16 shows a result of flow cytometric analysis of IL-17A and foxp3 for seven groups (Tₕ17, dNP2-ctCTLA-4 (WT), K1, K2 (G4S), K3 (G4S), K3 (GG) and K3 (no linker)) treated to Tₕ17 cells differentiated from splenocytes isolated from mouse.

FIG. 17 shows a result of flow cytometric analysis of CD4⁺IL-17A⁺ T cells and CD4⁺foxp3⁺ T cells for eight groups (Tₕ17, dNP2-ctCTLA-4 (WT), AP-ctCTLA-4, K1, K2 (G4S), K3 (G4S), K3 (GG) and K3 (no linker)).

FIG. 18 shows a result of evaluating clinical symptoms of a multiple sclerosis animal model for five groups (PBS, dNP2-ctCTLA-4 (WT), K1, K2 (G4S) and K3 (no linker)).

FIG. 19 shows a result of analyzing the number of immune cells (Tₕ1, Tₕ17 and T_{reg}) for five groups (PBS, dNP2-ctCTLA-4 (WT), K1, K2 (G4S) and K3 (no linker)).

FIG. 20 shows the structure of dNP2-ctCTLA-4 (WT), a K2 peptide of SEQ ID NO 1, a K3 peptide of SEQ ID NO 4, an AP-K2 peptide of SEQ ID NO 9 and an AP-K3 peptide of SEQ ID NO 10.

FIG. 21 shows a result of flow cytometric analysis of IL-17A and foxp3 for six groups (Tₕ17 (control group), dNP2-ctCTLA-4 (WT), K2, K3, AP-K2 and AP-K3) treated to Tₕ17 cells differentiated from splenocytes isolated from mouse.

FIG. 22 shows a result of evaluating clinical symptoms of a multiple sclerosis animal model for six groups (PBS (control group), dNP2-ctCTLA-4, dNP2, AP, AP-K2 and AP-K3).

FIG. 23 shows a result of analyzing the number of infiltrated lymphocytes and immune cells (T_{reg}) for six groups (PBS (control group), dNP2-ctCTLA-4, dNP2, AP, AP-K2 and AP-K3).

FIG. 24 shows the structure of dNP2-ctCTLA-4 (WT), a K2 peptide of SEQ ID NO 1, a K3 peptide of SEQ ID NO 4, an AP-K2 peptide of Example 15 and an AP-K3 peptide of Example 16.

FIG. 25 shows a result of flow cytometric analysis of IL-17A and foxp3 for six groups (Tₕ17(control group), dNP2-ctCTLA-4, K2, K3, Myr-K2 and Myr-K3) treated to Tₕ17 cells differentiated from splenocytes isolated from mouse.

FIG. 26 shows a result of evaluating clinical symptoms of a multiple sclerosis animal model for six groups (Tₕ17, dNP2-ctCTLA-4, K2, AP-K2, Myr-K2 and Myr-K3).

FIG. 27 shows a result of analyzing the number of infiltrated lymphocytes and immune cells (T_{reg}) for six groups (Tₕ17, dNP2-ctCTLA-4, K2, AP-K2, Myr-K2 and Myr-K3).

FIG. 28 shows a result of flow cytometric analysis of IL-17A and foxp3 for seven groups (Tₕ17, dNP2-ctCTLA-4, Myr-K3, Myr-K3D, Pal-K3, Myr-Ap-K2D and Pal-Ap-K2D) treated to Tₕ17 cells differentiated from splenocytes isolated from mouse.

FIG. 29 shows a result of flow cytometric analysis of IL-17A and foxp3 for eleven groups (Tₕ17, AP-K2, AP, AP-K2 KA, AP-K2 IKA, AP-K2 MA, AP-K2 LA, AP-K2 2KA, AP-K2 3KA, AP-K2 RA and AP-K2 SA) treated to Tₕ17 cells differentiated from splenocytes isolated from mouse.

FIGS. 30A and 30B show a result of flow cytometric analysis of CD4⁺IL-17A⁺ T cells (FIG. 30A) and CD4⁺foxp3⁺ T cells (FIG. 30B) for eleven groups (Tₕ17, AP-K2, AP, AP-K2 KA, AP-K2 IKA, AP-K2 MA, AP-K2 LA, AP-K2 2KA, AP-K2 3KA, AP-K2 RA and AP-K2 SA).

### BEST MODE

Hereinafter, the present disclosure will be described in more detail through examples. These examples are only for illustrating the present disclosure more specifically, and it will be obvious to those having ordinary knowledge in the art that the scope of the present disclosure is not limited by the examples.

### Examples 1-14. Synthesis of Peptides

Peptides of SEQ ID NOS 1-14 were designed as described in Table 1 and synthesized using solid-phase peptide synthesis (SPPS) by AnyGen (Kwangju, Korea). Briefly, amino acids protected by Fmoc were sequentially conjugated to a trityl chloride resin, and then the protecting groups and amino acid residues were removed from the resin through the global cleavage method to obtain crude peptides under acidic conditions. The crude peptides were purified by high-performance liquid chromatography (HPLC; Shimadzu, Kyoto, Japan). The finally synthesized peptides were freeze-dried and stored. The purity of the peptides was 95% or higher (AnyGen, Kwangju, Korea). The sequences of the synthesized fourteen peptides are as follows.

TABLE 1

| | | SEQ | |
|---|---|---|---|
| | | ID | |
| | Name | NO | Sequence |
| Example 1 | K2 | 1 | KMLKKRSKMLKKRS |
| Example 2 | K2 (G4S) | 2 | KMLKKRSGGGGSKMLKKRS |
| Example 3 | K2(GG) | 3 | KMLKKRSGGKMLKKRS |
| Example 4 | K3 | 4 | KMLKKRSKMLKKRSKMLKKRS |
| Example 5 | K3 (G4S) | 5 | KMLKKRSGGGGSKMLKKRSGGGGSKM |
| | | | LKKRS |
| Example 6 | K3 (GG) | 6 | KMLKKRSGGKMLKKRSGGKMLKKRS |
| Example 7 | dNP2-K2 | 7 | KIKKVKKKGRKGSKIKKVKKKGRKKMLK |
| | | | KRSKMLKKRS |
| Example 8 | dNP2-K3 | 8 | KIKKVKKKGRKGSKIKKVKKKGRKKMLK |
| | | | KRSKMLKKRSKMLKKRS |
| Example 9 | AP-K2 | 9 | RRRWCKRRRGGKMLKKRSKMLKKRS |
| Example 10 | AP-K3 | 10 | RRRWCKRRRGGKMLKKRSKMLKKRSK |
| | | | MLKKRS |
| Example 11 | KD2 | 11 | kmLKKrsKMLKKrs |
| Example 12 | KD3 | 12 | kmLKKrsKMLKKrsKMLKKrs |
| Example 13 | AP-KD2 | 13 | rrRWCKRRRGGKMLKKrsKMLKKrs |
| Example 14 | AP-KD3 | 14 | rrRWCKRRRGGKMLKKrsKMLKKrsKML |
| | | | KKrs |

In Table 1, the lower case letters mean D-amino acids.

### Examples 15-20. Synthesis of Peptides Modified With Fatty Acids

The peptides of SEQ ID NOS 3, 4, 11, 12 and 13 were modified with fatty acids (Myr-K2, Myr-K3, Myr-K3D, Pal-K3, Myr-AP-K2D and Pal-AP-K2D). Briefly, the amino acids protected by Fmoc were sequentially conjugated to a trityl chloride resin, and then the protecting groups and amino acid residues were removed from the resin through the global cleavage method to obtain crude peptides under acidic conditions. Peptides modified with fatty acids were synthesized by mixing the peptides with reagents (HOBt, DIC and DMF) and then conducting reaction at room temperature for a day. The crude peptides were purified by high-performance liquid chromatography (HPLC; Shimadzu, Kyoto, Japan). The finally synthesized peptides were freeze-dried and stored. The purity of the peptides was 95% or higher (AnyGen, Kwangju, Korea).

### Experimental Method 1. Analysis of Effect Under Tₕ17 Differentiation Conditions

### Differentiation

Tₕ17 cells are a subset of CD4 T cells. The Tₕ17 cells are known to secrete a cytokine known as IL-17 and cause inflammation. This is the major cause of autoimmune disease. And, T_{reg} cells (regulatory T cells) are known to suppress and regulate inflammation by expressing the transcription factor called Foxp3. Accordingly, since it is thought that the effect of preventing and treating autoimmune disease may be achieved by regulating inflammation by inhibiting Tₕ17 cells (IL-17) and increasing T_{reg} (Foxp3) cells, the effect of the peptides of the present disclosure was investigated.

Undifferentiated CD4 T cells (naive CD4 T cells) were isolated from splenocytes of 6- to 8-week-old C57BL/6 mice using a mouse undifferentiated CD4+ T cell (naive CD4 T cell) isolation kit (Miltenyi Biotec, Auburn, CA, USA) according to the manufacturer's protocols. For RNAseq, the undifferentiated CD4 T cells (naive CD4 T cells) from the Foxp3-GFP mice were purified, differentiated into Tₕ17 cells and sorted into Foxp3⁺ and Foxp3⁻ populations using a cell sorter (FACS Aria cell sorter BD Biosciences, Franklin Lakes, NJ, USA). The purified undifferentiated CD4 T cells (naive CD4 T cells) were activated with plate-bound anti-CD3 (2 or 5 μg/mL, 553057; BD Pharmingen, La Jolla, CA) and anti-CD28 (2 or 5 μg/mL, 553294; BD Pharmingen, La Jolla, CA) antibodies and differentiated by treating with the following cytokine cocktails in a 96-well plate for 3 or 4 days: Th0; recombinant human TGF-β(R&D Systems) 0.25 ng/mL. Tₕ1; anti-IL-4 (11B11; 16-7041-85; eBioscience) 5 μg/mL, IL-2 (212-12; Peprotech, Rocky Hill, NJ) 50 U/mL, recombinant murine IL-12 (212-12; Peprotech) 2 ng/mL, recombinant human TGF-β (R&D Systems) for Tₕ1 0.25 ng/mL. Tₕ17; anti-IFN-γ (XMG1.2; 16-7311-85; eBioscience) 5 μg/mL, anti-IL-4 (11B11; 16-7041-85; eBioscience) 5 μg/mL, recombinant murine TGF-β (7666-MB, R&D Systems, Minneapolis, MN) 2 ng/mL, IL-6 (216-16; Peprotech) 30 ng/mL, IL-23 (1887-ML-101; R&D Systems) 20 ng/mL, IL-1β (401-ML-010; R&D Systems) 20 ng/mL, and IL-2 (Peprotech) 50 U/mL. T_{reg}; IL-2 (Peprotech) 50 U/mL and TGF-β (R&D Systems) 0.5 ng/mL.

The cells were cotreated with the cytokine cocktails and the samples (2 μM). 4 days later, they were analyzed by flow cytometry using IL-17 and Foxp3 fluorescent antibodies. In some experiments, 5 μM PKC-η pseudosubstrate inhibitor (sc-3096; Santa Cruz) or 5 μM ERK inhibitor (UO126, 662005; Sigma) were also added.

### Flow Cytometric Analysis

For flow cytometric analysis of living cells only, dead cells were excluded using the Zombie Aqua™ fixable viability kit (Biolegend) before staining with antibodies at room temperature for 10 minutes. After washing, surface proteins of the differentiated Tₕ1, Tₕ17 and T_{reg} cells were stained with monoclonal antibodies at 4° C. for 15 minutes. To determine intracellular cytokine levels, the cells were restimulated with a cell stimulation cocktail (plus protein transport inhibitors) (ThermoFisher) at 37° C. for 4 hours and then stained with surface marker antibodies. The cells were then fixed, permeabilized using a Foxp3/transcription factor staining buffer set (eBioscience) and stained with anti-mouse IFN-γ, anti-mouse IL-17 and anti-mouse Foxp3. The cells were analyzed using a FACS Canto II flow cytometer and the FlowJo software version 10.7.

### Statistics

Statistical analysis was conducted by t-test and two-way ANOVA using the PRISM software. P<0.5 was regarded as statistically significant.

### Experimental Method 2. Analysis of Efficiency in Multiple Sclerosis (MS) Animal Model

### Experimental Animals

8-week-old female C57BL/6 mice were purchased from Orient Bio (Daejeon, Korea). All animal experiments were conducted in compliance with the animal experiment operation regulations of the Institutional Animal Care and Use Committee of Hanyang University. The mice were acclimatized for 2 weeks before the experiment. The mice were raised in a breeding room maintained at temperature 22±2° C. and 40-60% humidity with free access to feed. The light and dark cycles were adjusted at 12-hour intervals.

### Induction of Multiple Sclerosis

Multiple sclerosis was induced by subcutaneous immunization using a MOG₃₅₋₅₅/CFA emulsion PTX kit (Hooke Labs, Lawrence, MA, USA) according to the manufacturer's protocol. The mice were anesthetized with isoflurane and MOG₃₅₋₅₅/CFA was subcutaneously injected bilaterally, followed by intraperitoneal injection of PTX (pertussis toxin) 2 hours and 24 hours later. The mice were randomly assigned to different groups after MOG immunization. Because paralysis due to immune response begins on day 7 after the injection of PTX, 100 μg (5 mg/kg) of the samples according to the present disclosure were intraperitoneally injected to the groups once a day from day 7. Each group was monitored every day after inducing multiple sclerosis and clinical symptoms were recorded. The clinical symptoms were evaluated based on the scoring system described in Table 2. The higher clinical scores indicate severer symptoms.

TABLE 2

| Score | Description |
|---|---|
| 0 | Normal behavior; no obvious signs of disease |
| 0.5 | Partially limp tail |
| 1.0 | Completely limp tail |
| 1.5 | Limp tail and waddling gait |
| 2.0 | Erratic gait; instability of hind limbs during walking (paralysis of |
| | one hind limb) |
| 2.5 | Complete paralysis of one hind limb and partial paralysis of the |
| | other hind limb |
| 3.0 | Paralysis of both hind limbs; difficulty in standing with hind limbs |
| 3.5 | Inability to move forelimbs normally (ascending paralysis) |
| 4.0 | All the limbs are paralyzed and become thinner and weaker |
| | (paralysis of trunk) |
| 4.5 | Moribund |
| 5.0 | Dead |

### Flow Cytometric Analysis and Histological Analysis

The mice were euthanized at the end of the experiments and lymphocytes were isolated from the spinal cords. The isolated spinal cords were treated at 37° C. with 1 mg/mL of DNase 1 (10104159001; Sigma-Aldrich) and 1 mg/mL of collagenase D (11088866001; Sigma-Aldrich) and incubated at 80 rpm on a shaker for 40 minutes. After the enzyme treatment, 500 mM EDTA was added and lymphocytes were isolated by Percoll (GE Healthcare, Little Chalfont, UK) density-gradient centrifugation. The isolated infiltrated lymphocytes were restimulated with a cell stimulation cocktail (plus protein transport inhibitors) (ThermoFisher) at 37° C. for 4 hours and stained with anti-mouse CD4. The cells were fixed, permeabilized using a Foxp3/transcription factor staining buffer set (eBioscience) and stained with anti-mouse IFN-γ, anti-mouse IL-17 or anti-mouse Foxp3. The cells were analyzed using a FACS Canto flow cytometer and the FlowJo software version 10.7.1.

For histological analysis, paraffin blocks of the spinal cord tissues were deparaffinized and treated with luxol fast blue. For combination staining, hematoxylin and eosin were used (Dako). The cells infiltrated in the white matter region of the spinal cord tissues were counted using the Image J software version 2.0.0.

The increased number of Tₕ1 and Tₕ17 immune cells in the spinal cord means more severe multiple sclerosis, and the increased number of T_{reg} cells means that the function of preventing or treating autoimmune disease such as multiple sclerosis is improved as the tolerance to self-antigens is maintained and the excessive response by the immune system is prevented.

### Statistics

Statistical analysis was conducted by t-test and two-way ANOVA using the PRISM software. P<0.5 was regarded as statistically significant.

### Test Example 1. Investigation of Function of dNP2-ctCTLA-4 Variants

### Preparation of Samples

The degree of suppression of immune response was analyzed to investigate the function of dNP2-ctCTLA-4 variants.

First, dNP2-ctCTLA-4 and dNP2-ctCTLA-4 variants (KA, PA and YF) were designed as described in Table 3 and synthesized using solid-phase peptide synthesis (SPPS) by AnyGen (Kwangju, Korea). Specific preparation processes are the same as described in Example 1. The purity of the prepared peptides was also 95% or higher.

TABLE 3

| | SEQ | |
|---|---|---|
| | ID | |
| Name | NO | Amino acid sequence |
| dNP2- | 15 | KIKKVKKKGRKGSKIKKVKKKGRKGGKMLKKRSPL |
| ctCTLA- | | TTGVYVKMPPTEPECEKQFQPYFIPIN |
| 4WT | | |
| dNP2-KA | 19 | KIKKVKKKGRKGSKIKKVKKKGRKGGAMLAAASPL |
| | | TTGVYVKMPPTEPECEKQFQPYFIPIN |
| dNP2-PA | 20 | KIKKVKKKGRKGSKIKKVKKKGRKGGKMLKKRSPL |
| | | TTGVYVKMAATEAECEKQFQPYFIPIN |
| dNP2-YF | 21 | KIKKVKKKGRKGSKIKKVKKKGRKGGKMLKKRSPL |
| | | TTGVFVKMPPTEPECEKQFQPFFIPIN |

### Sample Administration—Differentiation of Tₕ17 Cells

The efficacy depending on Tₕ17 differentiation condition was analyzed in the same manner as in Experimental Method 1 except that dNP2-ctCTLA-4 and dNP2-ctCTLA-4 variants (KA, PA and YF) were used as samples. For five groups (Tₕ17, dNP2-ctCTLA-4, KA, PA and YF), flow cytometry was conducted 4 days later using IL-17 and Foxp3 fluorescent antibodies.

Tₕ17 group (control group): CD4 T cells activated with anti-CD3 and anti-CD28 were treated with cytokine cocktail and PBS of the same volume as a positive control group.

dNP2-ctCTLA-4 group (positive control group): CD4 T cells activated with anti-CD3 and anti-CD28 were treated with cytokine cocktail and dNP2-ctCTLA-4 of SEQ ID NO 15 (2 μM).

KA group: CD4 T cells activated with anti-CD3 and anti-CD28 were treated with cytokine cocktail and dNP2-KA of SEQ ID NO 20 (2 μM).

PA group: CD4 T cells activated with anti-CD3 and anti-CD28 were treated with cytokine cocktail and dNP2-PA of SEQ ID NO 21 (2 μM).

YF group: CD4 T cells activated with anti-CD3 and anti-CD28 were treated with cytokine cocktail and dNP2-YF of SEQ ID NO 22 (2 μM).

### Sample Administration—Multiple Sclerosis Animal Model

The efficacy in a multiple sclerosis animal model was analyzed according to Experimental Method 2 using dNP2-ctCTLA-4 and dNP2-ctCTLA-4 variants (KA, PA and YF) as samples for five groups (PBS, WT, KA, PA and YF).

PBS group (control group): 100 μg of PBS was intraperitoneally injected once a day from day 7 to day 14 after the injection of PTX.

dNP2-ctCTLA-4 group (positive control group): (dNP2-ctCTLA-4)_WT of SEQ ID NO 15 (100 μg) was intraperitoneally injected once a day from day 7 to day 14 after the injection of PTX.

KA group: dNP2-KA peptide of SEQ ID NO 20 (100 μg) was intraperitoneally injected once a day from day 7 to day 14 after the injection of PTX.

PA group: dNP2-PA peptide of SEQ ID NO 21 (100 μg) was intraperitoneally injected once a day from day 7 to day 14 after the injection of PTX.

YF group: dNP2-YF peptide of SEQ ID NO 22 (100 μg) was intraperitoneally injected once a day from day 7 to day 14 after the injection of PTX.

After the induction of multiple sclerosis, each group was monitored every day as in Experimental Method 2 and clinical score was recorded. After the experiment was completed, lymphocytes, CD4⁺IL-17A⁺ T cells, CD4⁺IFN-γ⁺ T cells and T_{reg}/T_{eff} ratio were measured through histological analysis.

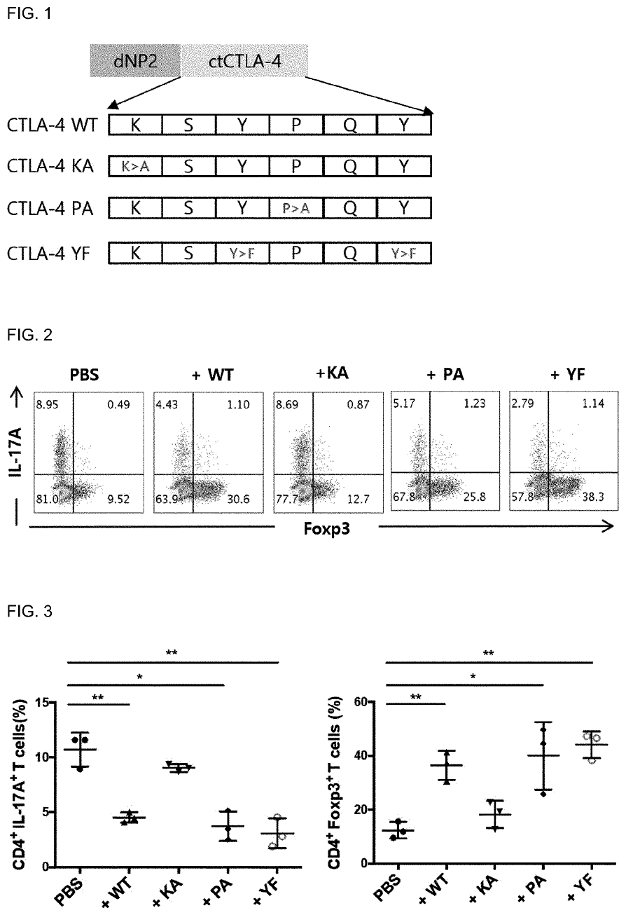

FIG. 1 shows the structure of dNP2-ctCTLA-4 and dNP2-ctCTLA-4 variants (KA, PA and YF), FIG. 2 shows a result of flow cytometric analysis of IL-17A and foxp3 for five groups (Tₕ17, WT, KA, PA and YF) treated to Tₕ17 cells differentiated from splenocytes isolated from mouse, and FIG. 3 shows a result of flow cytometric analysis of CD4⁺IL-17A⁺ T cells and CD4⁺foxp3⁺ T cells for five groups (Th17, WT, KA, PA and YF) prepared using dNP2-ctCTLA-4 and dNP2-ctCTLA-4 variants (KA, PA and YF).

As shown in FIG. 1, the correlation with the efficacy of suppressing immune response was analyzed for the ‘K, S, Y, P, Q and Y’ motifs of the cytoplasmic domain of CTLA4.

As shown in FIGS. 2 and 3, it was confirmed that, when treated with the KA peptide of SEQ ID NO 20 in which some amino acid residues of the K motif were replaced with alanine residues (KA group), the function of the decrease of IL-17 and the increase of Foxp3 was lost unlike other groups. Accordingly, it was confirmed that the K motif in CTLA4 is highly related to the suppression of immune response.

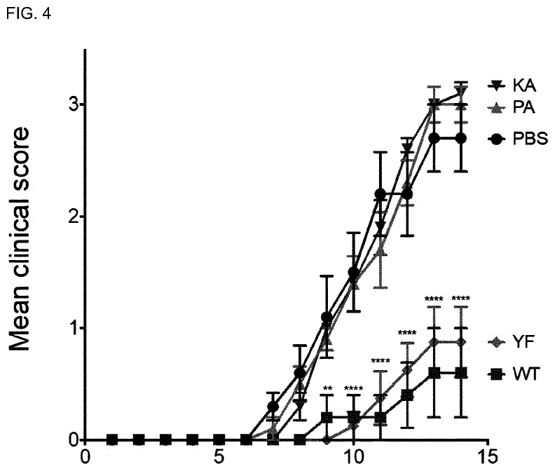

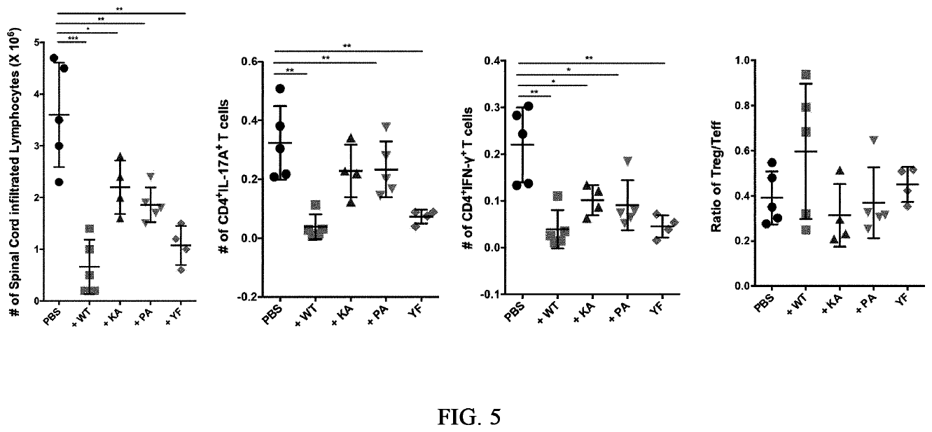

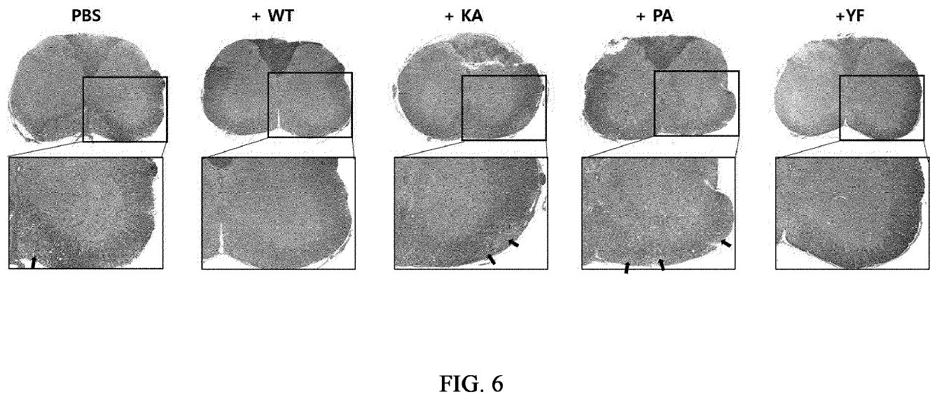

FIG. 4 shows a result of evaluating clinical symptoms of a multiple sclerosis animal model for five groups (PBS, WT, KA, PA and YF) prepared using dNP2-ctCTLA-4 and dNP2-ctCTLA-4 variants (KA, PA and YF), FIG. 5 shows a result of analyzing the number of immune cells (Tₕ1, Tₕ17 and T_{reg}) for five groups (PBS, WT, KA, PA and YF) prepared using dNP2-ctCTLA-4 and dNP2-ctCTLA-4 variants (KA, PA and YF), FIG. 6 shows a result of histological analysis of the spinal cord of a multiple sclerosis animal model to which five groups (PBS, WT, KA, PA and YF) prepared using dNP2-ctCTLA-4 and dNP2-ctCTLA-4 variants (KA, PA and YF) were administered.

As shown in FIG. 4 and FIG. 5, the KA group and the PA group showed significantly lower clinical symptoms as compared to the PBS group (control group). In addition, the KA group and the PA group showed significantly larger number of infiltrated lymphocytes in the spinal cord as compared to the PBS group (control group). In particular, the KA group showed the smallest number of T_{reg} cells. Taken together, it can be seen that the K motif in CTLA4 plays a major role in suppressing immune response in autoimmune disease.

As shown in FIG. 6, as a result of histological analysis of the spinal cords of the five groups (PBS, WT, KA, PA and YF) in the multiple sclerosis animal model, it was confirmed that the function of inhibiting myelin damage was lost in the KA group, indicating that the K motif plays a major role in autoimmune disease.

### Test Example 2. Analysis of Efficacy of Deleted Variants of CTLA4

### Sample Preparation

In order to reconfirm the function of the K motif in dNP2-ctCTLA-4, deleted variants were prepared by deleting motifs from dNP2-ctCTLA-4 and the inhibition of immune response was analyzed.

First, deleted variants of dNP2-ctCTLA-4, dNP2 and dNP2-ctCTLA-4 (KSYPQ, KSYP, KSY, KS and K) were designed as described in Table 4 and synthesized by AnyGen (Kwangju, Korea) using solid-phase peptide synthesis (SPPS). Specific procedures were the same as described in Example 1 and the prepared peptides also showed purity of 95% or higher.

TABLE 4

| | SEQ | |
|---|---|---|
| | ID | |
| Name | NO | Amino acid sequence |
| (dNP2- | 15 | KIKKVKKKGRKGSKIKKVKKKGRKGGKMLKKRS |
| ctCTLA-4) | | PLTTGVYVKMPPTEPECEKQFQPYFIPIN |
| WT | | |
| (TAT- | 16 | YGRKKRRRQRRRGSKIKKVKKKGRKGGKMLKK |
| ctCTLA-4) | | RSPLTTGVYVKMPPTEPECEKQFQPYFIPIN |
| WT | | |
| dNP2 | 18 | KIKKVKKKGRKGSKIKKVKKKGRK |
| dNP2-KSYPQ | 23 | KIKKVKKKGRKGSKIKKVKKKGRKGGKMLKKRS |
| | | PLTTGVYVKMPPTEPECEKQFQP |
| dNP2-KSYP | 24 | KIKKVKKKGRKGSKIKKVKKKGRKGGKMLKKRS |
| | | PLTTGVYVKMPPTEPECE |
| dNP2-KSY | 25 | KIKKVKKKGRKGSKIKKVKKKGRKGGKMLKKRS |
| | | PLTTGVYVKM |
| dNP2-KS | 26 | KIKKVKKKGRKGSKIKKVKKKGRKGGKMLKKRS |
| | | PLTTGV |
| dNP2-K1 | 27 | KIKKVKKKGRKGSKIKKVKKKGRKKMLKKRS |

### Sample Administration—Tₕ17 Differentiation Condition

The efficacy depending on Tₕ17 differentiation condition was analyzed in the same manner as in Experimental Method 1 except that deleted variants of dNP2-ctCTLA-4, dNP2 and dNP2-ctCTLA-4 (KSYPQ, KSYP, KSY, KS and K) were used as samples. Flow cytometry was conducted using IL-17 and Foxp3 fluorescent antibodies 4 days later for eight groups (Tₕ17, dNP2, dNP2-ctCTLA-4, KSYPQ, KSYP, KSY, KS and K).

Tₕ17 group (control group 1): CD4 T cells activated with anti-CD3 and anti-CD28 were treated with cytokine cocktail and PBS (2 μM).

dNP2 group (control group 2): CD4 T cells activated with anti-CD3 and anti-CD28 were treated with cytokine cocktail and dNP2 peptide of SEQ ID NO 18 (2 μM).

dNP2-ctCTLA-4 group (positive control group): CD4 T cells activated with anti-CD3 and anti-CD28 were treated with cytokine cocktail and dNP2-ctCTLA-4 of SEQ ID NO 15 (2 μM).

KSYPQ group: CD4 T cells activated with anti-CD3 and anti-CD28 were treated with cytokine cocktail and dNP2-KSYPQ peptide of SEQ ID NO 23 (2 μM).

KSYP group: CD4 T cells activated with anti-CD3 and anti-CD28 were treated with cytokine cocktail and dNP2-KSYP peptide of SEQ ID NO 24 (2 μM).

KSY group: CD4 T cells activated with anti-CD3 and anti-CD28 were treated with cytokine cocktail and dNP2-KSY peptide of SEQ ID NO 25 (2 μM).

KS group: CD4 T cells activated with anti-CD3 and anti-CD28 were treated with cytokine cocktail and dNP2-KS peptide of SEQ ID NO 26 (2 μM).

K group: CD4 T cells activated with anti-CD3 and anti-CD28 were treated with cytokine cocktail and dNP2-K1 peptide of SEQ ID NO 27 (2 μM).

### Sample Administration—Multiple Sclerosis Animal Model

The efficacy in a multiple sclerosis animal model was analyzed according to Experimental Method 2 for five groups (PBS, dNP2-ctCTLA-4, TAT-ctCTLA-4, KSY and K) using dNP2-ctCTLA-4, TAT-ctCTLA-4 and deleted variants of dNP2-ctCTLA-4 (KSY and K).

PBS group (control group): 100 μg of PBS was intraperitoneally injected once a day from day 7 to day 15 after the injection of PTX.

dNP2-ctCTLA-4 group (positive control group 1): dNP2-ctCTLA-4_WT of SEQ ID NO 15 (100 μg) was intraperitoneally injected once a day from day 7 to day 15 after the injection of PTX.

TAT-ctCTLA-4 group (positive control group 2): A TAT-ctCTLA-4 peptide of SEQ ID NO 16 (100 μg) was intraperitoneally injected once a day from day 7 to day 15 after the injection of PTX.

dNP2-KSY group: A dNP2-KSY peptide of SEQ ID NO 25 (100 μg) was intraperitoneally injected once a day from day 7 to day 15 after the injection of PTX.

dNP2-K group: A dNP2-K1 peptide of SEQ ID NO 27 (100 μg) was intraperitoneally injected once a day from day 7 to day 15 after the injection of PTX.

After the induction of multiple sclerosis, each group was monitored every day as in Experimental Method 2 and clinical score was recorded. After the experiment was completed, lymphocytes, CD4⁺IL-17A⁺ T cells, CD4⁺IFN-γ⁺ T cells and T_{reg}/T_{eff} ratio were measured.

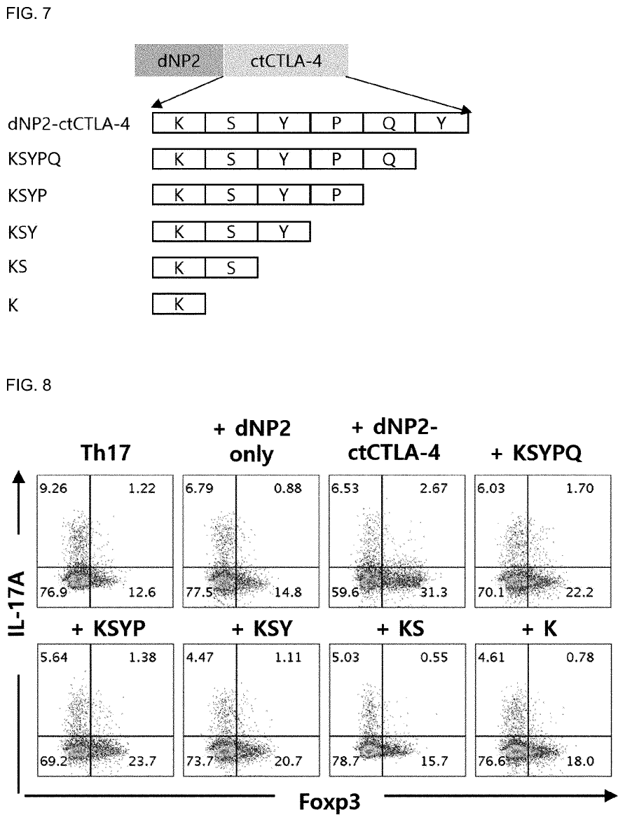

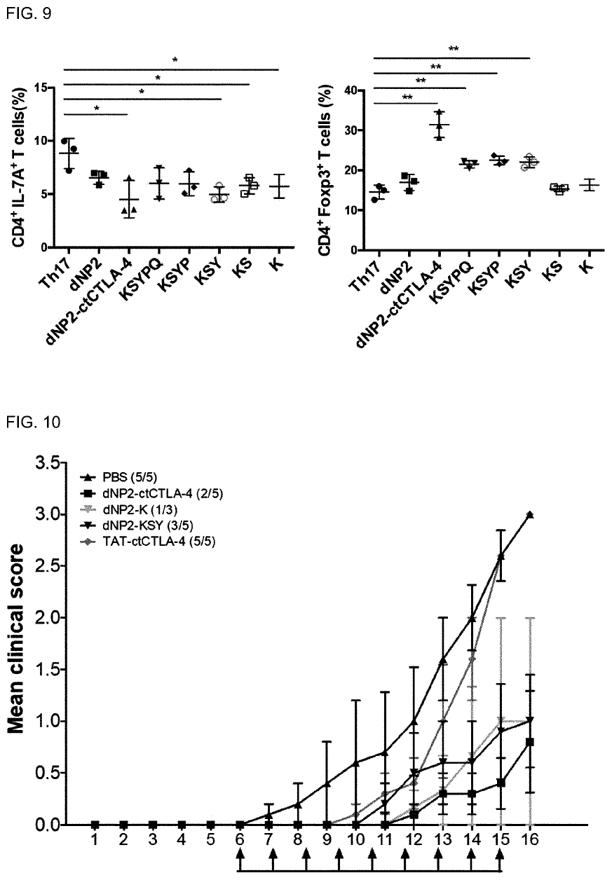

FIG. 7 shows the structure of deleted variants of dNP2-ctCTLA-4, dNP2 and dNP2-ctCTLA-4 (KSYPQ, KSYP, KSY, KS and K), FIG. 8 shows a result of flow cytometric analysis of IL-17A and foxp3 for eight groups (Tₕ17, dNP2, dNP2-ctCTLA4, KSYPQ, KSYP, KSY, KS and K) treated to Tₕ17 cells differentiated from splenocytes isolated from mouse, and FIG. 9 shows a result of flow cytometric analysis of CD4⁺IL-17A⁺ T cells and CD4⁺foxp3⁺ T cells for eight groups (Tₕ17, dNP2, dNP2-ctCTLA4, KSYPQ, KSYP, KSY, KS and K).

As shown in FIG. 7, for more accurate analysis of the performance of the K motif, the efficacy of suppressing immune response was analyzed while removing ‘K, S, Y, P, Q and Y’ motifs one by one.

As shown in FIGS. 8 and 9, the dNP2-K1 peptide of SEQ ID NO 27, in which only the K motif is present in dNP2-ctCTLA-4 (K group), exhibited the activity of inhibiting immune response. Through this, it can be seen that the K motif in dNP2-ctCTLA-4 plays a major role in the suppression of immune response.

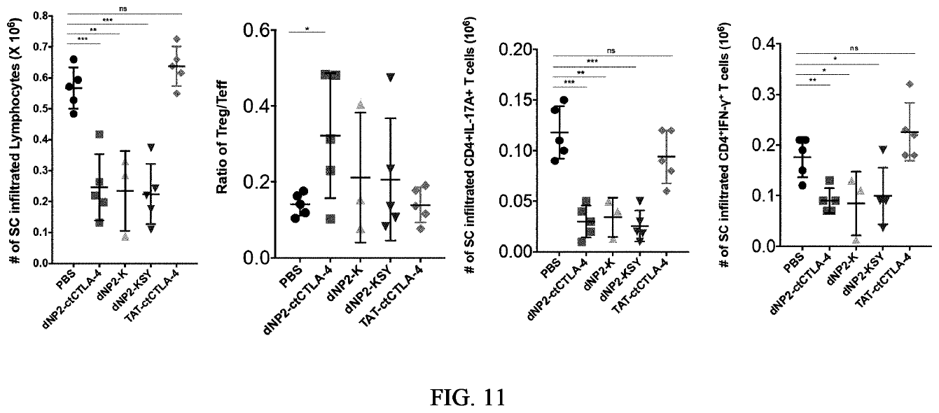

FIG. 10 shows a result of evaluating clinical symptoms of a multiple sclerosis animal model for five groups (PBS, dNP2-ctCTLA-4, TAT-ctCTLA-4, KSY and K), and FIG. 11 shows a result of analyzing the number of immune cells (Tₕ1, Tₕ17 and T_{reg}) for five groups (PBS, dNP2-ctCTLA-4, TAT-ctCTLA-4, KSY and K).

As shown in FIG. 10 and FIG. 11, it was confirmed that the KSY group and the K group was significantly superior in terms of clinical symptoms as compared to the TAT-ctCTLA-4 group and the PBS group. In addition, the dNP2-KSY group and the dNP2-K group had significantly smaller number of infiltrated lymphocytes in the spinal cord as compared to the TAT-ctCTLA-4 group and the PBS group. That is to say, it was confirmed that superior activity of suppressing immune response is achieved only with the K motif.

### Test Example 3. Analysis of Efficacy of Motifs of CTLA4

### Sample Preparation

In order to investigate the function of dNP2-ctCTLA-4, dNP2 and dNP2-ctCTLA-4 variants (K, S, Y1, P and Y2), the suppression of immune response was analyzed for dNP2-ctCTLA-4 and its variants.

First, dNP2-ctCTLA-4 (WT), dNP2 and dNP2-ctCTLA-4 variants (K, S, Y1, P and Y2) were designed as described in Table 5 and were synthesized by AnyGen (Kwangju, Korea) using solid-phase peptide synthesis (SPPS). Specific preparation procedures were the same as in Example 1 and the prepared peptides also had a purity of 95% or higher.

TABLE 5

| | SEQ | |
|---|---|---|
| | ID | |
| Name | NO | Amino acid sequence |
| dNP2- | 15 | KIKKVKKKGRKGSKIKKVKKKGRKGGKMLKKRS |
| ctCTLA-4 | | PLTTGVYVKMPPTEPECEKQFQPYFIPIN |
| dNP2 | 18 | KIKKVKKKGRKGSKIKKVKKKGRK |
| K1 | 28 | KMLKKRS |
| S | 29 | SPLTTGV |
| Y1 | 30 | YVKM |
| P | 31 | PPTEPECE |
| Y2 | 32 | YFIPIN |

### Sample Administration—Tₕ17 Differentiation Condition

The efficacy under Tₕ17 differentiation conditions was analyzed in the same manner as in Experimental Method 1 except that dNP2-ctCTLA-4, dNP2 and dNP2-ctCTLA-4 variants (K, S, Y1, P and Y2) were used as samples. Flow cytometry was conducted 4 days later for eight groups (Tₕ17, dNP2, dNP2-ctCTLA-4, K, S, Y1, P and Y2) using IL-17 and Foxp3 fluorescent antibodies.

Tₕ17 group (control group): CD4 T cells activated with anti-CD3 and anti-CD28 were treated with cytokine cocktail and PBS (2 μM).

dNP2 group (control group 2): CD4 T cells activated with anti-CD3 and anti-CD28 were treated with cytokine cocktail and dNP2 peptide of SEQ ID NO 18 (2 μM).

dNP2-ctCTLA-4 group (positive control group): CD4 T cells activated with anti-CD3 and anti-CD28 were treated with cytokine cocktail and (dNP2-ctCTLA-4)_WT of SEQ ID NO 15 (2 μM).

K group: CD4 T cells activated with anti-CD3 and anti-CD28 were treated with cytokine cocktail and K1 peptide of SEQ ID NO 28 (2 μM).

S group: CD4 T cells activated with anti-CD3 and anti-CD28 were treated with cytokine cocktail and S peptide of SEQ ID NO 29 (2 μM).

Y1 group: CD4 T cells activated with anti-CD3 and anti-CD28 were treated with cytokine cocktail and Y1 peptide of SEQ ID NO 30 (2 μM).

P group: CD4 T cells activated with anti-CD3 and anti-CD28 were treated with cytokine cocktail and P peptide of SEQ ID NO 31 (2 μM).

Y2 group: CD4 T cells activated with anti-CD3 and anti-CD28 were treated with cytokine cocktail and Y2 peptide of SEQ ID NO 32 (2 μM).

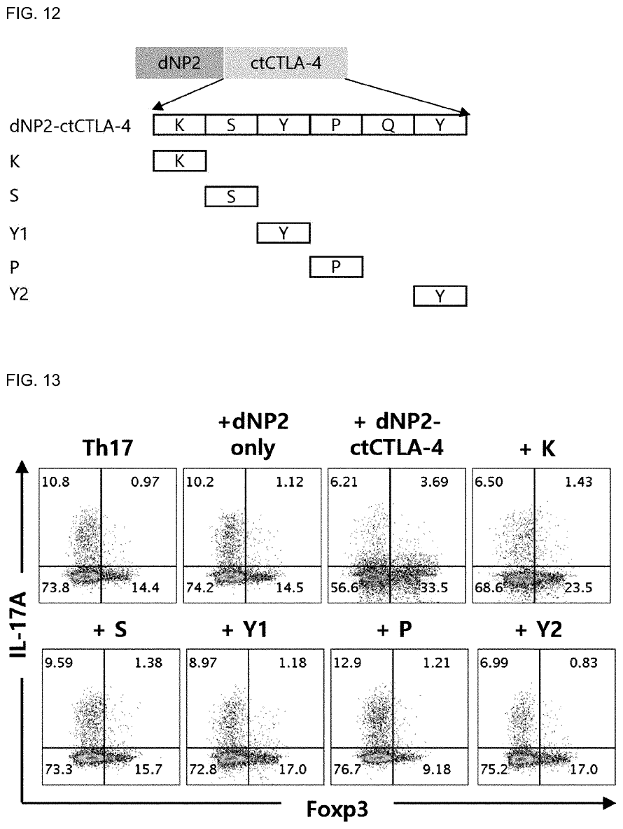

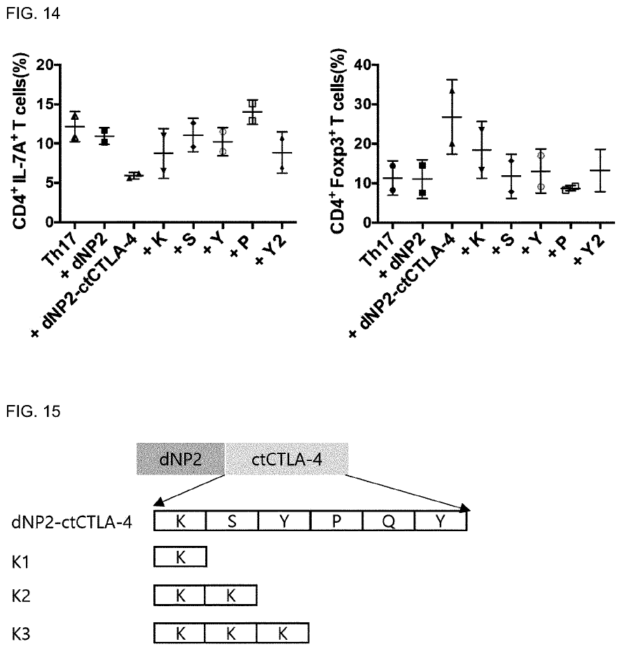

FIG. 12 shows the structure of dNP2-ctCTLA-4 (WT), dNP2, dNP2-ctCTLA-4 variants (K, S, Y1, P and Y2), FIG. 13 shows a result of flow cytometric analysis of IL-17A and foxp3 for eight groups (Tₕ17, dNP2, dNP2-ctCTLA-4 (WT), K, S, Y1, P and Y2) treated to Tₕ17 cells differentiated from splenocytes isolated from mouse, and FIG. 14 shows a result of flow cytometric analysis of CD4⁺IL-17A⁺ T cells and CD4⁺foxp3⁺ T cells for eight groups (Tₕ17, dNP2, dNP2-ctCTLA-4, K, S, Y1, P and Y2).

As shown in FIGS. 13 and 14, the K group consisting only of the K motif showed the activity of inhibiting immune response similar to that of the dNP2-ctCTLA4 group. Other groups, i.e., the S group, Y1 group, P group and Y2 group, did not show significant difference from the dNP2 group or the Tₕ17 group. Therefore, it was confirmed that the K motif plays a major role in the suppression of immune response.

### Test Example 4. Analysis of Efficacy for K, K2 and K3 Peptides

### Sample Preparation

In order to investigate the function of the K motif of dNP2-ctCTLA-4, the degree of suppression of immune response by the K motif was analyzed.

First, dNP2-ctCTLA-4, a K1 peptide of SEQ ID NO 28 and K variants of SEQ ID NOS 2 and 4-6 (K2 (G4S), K3 (G4S), K3 (GG) and K3) were designed as described in Table 6. The dNP2-ctCTLA-4 (WT) and the K1 peptide of SEQ ID NO 28 were synthesized by AnyGen (Kwangju, Korea) using solid-phase peptide synthesis (SPPS). The specific preparation procedure was the same as in Example 1 and Example 2, Example 4, Example 5 and Example 6 were used as the K variants of SEQ ID NOS 2 and 4-6 (K2 (G4S), K3 (G4S), K3 (GG) and K3). The prepared peptides also showed purity of 95% or higher.

The ‘K1’ peptide is a peptide represented by SEQ ID NO 28 with no substitution, addition or deletion of D-or L-amino acids and with no addition of a fatty acid or a linker at the terminal.

TABLE 6

| | SEQ | |
|---|---|---|
| | ID | |
| Name | NO | Amino acid sequence |
| dNP2- | 15 | KIKKVKKKGRKGSKIKKVKKKGRKGGKMLKK |
| ctCTLA-4 | | RSPLTTGVYVKMPPTEPECEKQFQPYFIPIN |
| K1 | 28 | KMLKKRS |
| K2 (G4S) | 2 | KMLKKRSGGGGSKMLKKRS |
| (Example 2) | | |
| K3 (no | 4 | KMLKKRSKMLKKRSKMLKKRS |
| linker) | | |
| (Example 4) | | |
| K3 (G4S) | 5 | KMLKKRSGGGGSKMLKKRSGGGGSKMLKKRS |
| (Example 5) | | |
| K3 (GG) | 6 | KMLKKRSGGKMLKKRSGGKMLKKRS |
| (Example 6) | | |

### Sample Administration—Tₕ17 Differentiation Condition

The efficacy under Tₕ17 differentiation conditions was analyzed in the same manner as in Experimental Method 1 except that dNP2-ctCTLA-4, the K1 peptide of SEQ ID NO 28, K1 variants of SEQ ID NOS 2 and 4-6 (K2 (G4S), K3 (G4S), K3 (GG) and K3) were used as samples. Flow cytometry was conducted using IL-17 and Foxp3 fluorescent antibodies 4 days later for eight groups (Tₕ17, dNP2-ctCTLA-4, K1, K2 (G4S), K3 (G4S), K3 (GG) and K3 (no linker)).

Tₕ17 group (control group): CD4 T cells activated with anti-CD3 and anti-CD28 were treated with cytokine cocktail and PBS (2 μM).

dNP2-ctCTLA-4 group (positive control group 1): CD4 T cells activated with anti-CD3 and anti-CD28 were treated with cytokine cocktail and the dNP2-ctCTLA-4 of SEQ ID NO 15 (2 μM).

AP-ctCTLA-4 group (positive control group 2): CD4 T cells activated with anti-CD3 and anti-CD28 were treated with cytokine cocktail and the AP-ctCTLA-4 of SEQ ID NO 17 (2 μM).

K1 group: CD4 T cells activated with anti-CD3 and anti-CD28 were treated with cytokine cocktail and the K1 peptide of SEQ ID NO 28 (2 μM).

K2 (G4S) group: CD4 T cells activated with anti-CD3 and anti-CD28 were treated with cytokine cocktail and the K2 (G4S) peptide of SEQ ID NO 2 (2 μM).

K3 (no linker) group: CD4 T cells activated with anti-CD3 and anti-CD28 were treated with cytokine cocktail and the K3 (no linker) peptide of SEQ ID NO 4 (2 μM).

K3 (G4S) group: CD4 T cells activated with anti-CD3 and anti-CD28 were treated with cytokine cocktail and the K3 (G4S) peptide of SEQ ID NO 5 (2 μM).

K3 (GG) group: CD4 T cells activated with anti-CD3 and anti-CD28 were treated with cytokine cocktail and the K3 (GG) peptide of SEQ ID NO 6 (2 μM).

### Sample Administration—Multiple Sclerosis Animal Model

The efficacy in a multiple sclerosis animal model was analyzed according to Experimental Method 2 for five groups (Tₕ17, dNP2-ctCTLA-4, K1, K2 (G4S) and K3 (no linker)) using dNP2-ctCTLA-4, the K1 peptide of SEQ ID NO 28, the K2 peptide of SEQ ID NO 2 and the K3 (no linker) peptide of SEQ ID NO 4 as samples.

PBS group (control group): 100 μg of PBS was intraperitoneally injected once a day from day 7 to day 18 after the injection of PTX.

dNP2-ctCTLA-4 group (positive control group 1): The dNP2-ctCTLA-4_WT of SEQ ID NO 15 (100 μg) was intraperitoneally injected once a day from day 7 to day 18 after the injection of PTX.

K1 group (positive control group 2): The K1 peptide of SEQ ID NO 28 (100 μg) was intraperitoneally injected once a day from day 7 to day 18 after the injection of PTX.

K2 (G4S) group: The K2 (G4S) peptide of SEQ ID NO 2 (100 μg) was intraperitoneally injected once a day from day 7 to day 18 after the injection of PTX.

K3 (no linker) group: The K3 peptide of SEQ ID NO 4(100 μg) was intraperitoneally injected once a day from day 7 to day 18 after the injection of PTX.

After the induction of multiple sclerosis, each group was monitored every day as in Experimental Method 2 and clinical score was recorded. After the experiment was completed, lymphocytes, CD4⁺IL-17A⁺ T cells, CD4⁺IFN-γ⁺ T cells and T_{reg}/T_{eff} ratio were measured through histological analysis.

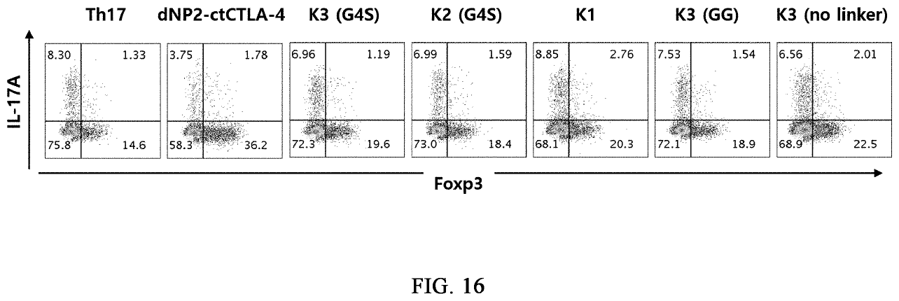

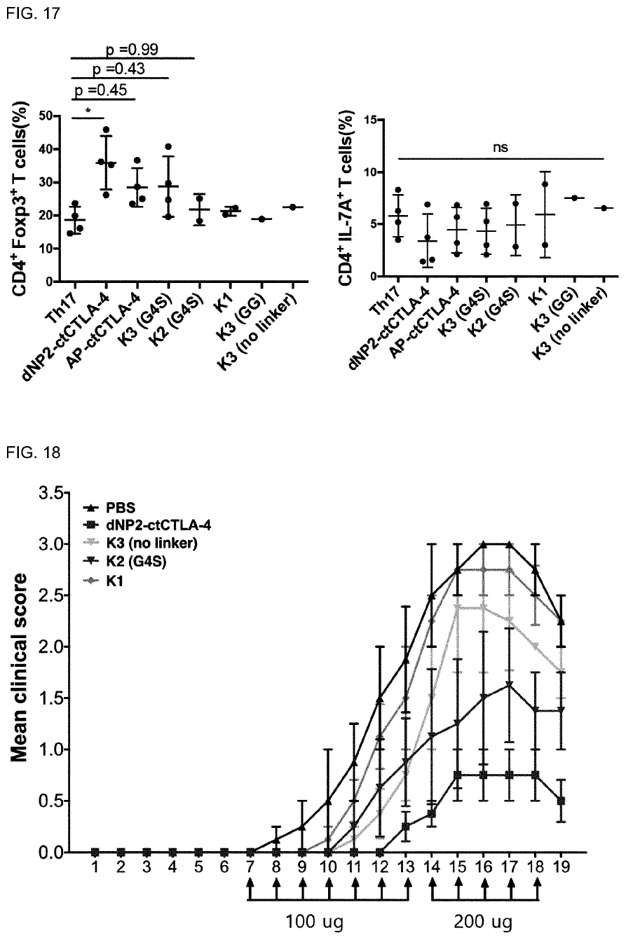

FIG. 15 shows the structure of dNP2-ctCTLA-4 (WT), K1, K2 (G4S), K3 (G4S), K3 (GG) and K3 (no linker), FIG. 16 shows a result of flow cytometric analysis of IL-17A and foxp3 for seven groups (Tₕ17, dNP2-ctCTLA-4 (WT), K1, K2 (G4S), K3 (G4S), K3 (GG) and K3 (no linker)) treated to Tₕ17 cells differentiated from splenocytes isolated from mouse, and FIG. 17 shows a result of flow cytometric analysis of CD4⁺IL-17A⁺ T cells and CD4⁺foxp3⁺ T cells for eight groups (Tₕ17, dNP2-ctCTLA-4 (WT), AP-ctCTLA-4, K1, K2 (G4S), K3 (G4S), K3 (GG) and K3 (no linker)).

As shown in FIG. 16 and FIG. 17, it can be seen that the activity of inhibiting immune cells is significantly superior when the peptides wherein the cell-penetrating peptide (CPP) such as dNP2 was removed and two or three of the K motifs were linked were used (Example 2, Example 4), as compared to the K1 group. That is to say, it was confirmed that, unlike dNP2-ctCTLA4, the K2 peptide and the K3 peptide show remarkably superior effect of suppressing immune response even without the CPP. In contrast, the K1 peptide did not show significant difference from Tₕ17 (control group) without the CPP.

Accordingly, it can be seen that the effect of preventing or treating autoimmune disease cannot be achieved with the K1 peptide although it is derived from dNP2-ctCTLA4.

FIG. 18 shows a result of evaluating clinical symptoms of a multiple sclerosis animal model for five groups (PBS, dNP2-ctCTLA-4 (WT), K1, K2 (G4S) and K3 (no linker)), and FIG. 19 shows a result of analyzing the number of immune cells (Tₕ1, Tₕ17 and T_{reg}) for five groups (PBS, dNP2-ctCTLA-4 (WT), K1, K2 (G4S) and K3 (no linker)).

As shown in FIG. 18 and FIG. 19, the K2 (G4S) group and the K3 (no linker) group showed significantly remarkable superior result in clinical symptoms than the K1 group. In addition, the K2 (G4S) group and the K3 (no linker) group showed significantly number of infiltrated lymphocytes in the spinal cord as compared to the K1 group. In particular, the K2 (G4S) group and the K3 (no linker) group showed larger number of T_{reg} cells than the K1 group. That is to say, it can be seen that, although the K motif does not show significant effect on its own in the multiple sclerosis animal model, the K2 and K3 peptides shows remarkably significant effect for multiple sclerosis in vivo.

### Test Example 5. Analysis of Efficacy of AP-K2 and AP-K3 Peptides

### Sample Preparation

The efficacy of CPP-bound K2 and K3 peptide complexes was analyzed. For this, dNP2-ctCTLA-4, K2, K3 (no linker), AP-K2 and AP-K3 were prepared as in Example 1, Example 4, Example 9 and Example 10. Their sequences are described in Table 7.

TABLE 7

| SEQ | | |
|---|---|---|
| ID | | |
| NO | Name | Amino acid sequence |
| 15 | dNP2- | KIKKVKKKGRKGSKIKKVKKKGRKGGKMLKKRSPLT |
| | ctCTLA-4 | TGVYVKMPPTEPECEKQFQPYFIPIN |
| 18 | dNP2 | KIKKVKKKGRKGSKIKKVKKKGRK |
| 19 | AP | RRRWCKRRR |
| 1 | K2 | KMLKKRSKMLKKRS |
| 4 | K3 | KMLKKRSKMLKKRSKMLKKRS |
| 9 | AP-K2 | RRRWCKRRRGGKMLKKRSKMLKKRS |
| 10 | AP-K3 | RRRWCKRRRGGKMLKKRSKMLKKRSKMLKKRS |

### Sample Administration—Tₕ17 Differentiation Condition

The efficacy under Tₕ17 differentiation conditions was analyzed in the same manner as in Experimental Method 1 except that dNP2-ctCTLA-4, the K2 peptide of SEQ ID NO 1, the K3 peptide of SEQ ID NO 4, the AP-K2 peptide of SEQ ID NO 9 and the AP-K3 peptide of SEQ ID NO 10 were used as samples. Experiment was conducted for six groups (Tₕ17 (control group), dNP2-ctCTLA-4, K2, K3, AP-K2 and AP-K3).

Tₕ17 group (control group 1): CD4 T cells activated with anti-CD3 and anti-CD28 were treated with cytokine cocktail and PBS (2 μM).

dNP2-ctCTLA-4 group (positive control group): CD4 T cells activated with anti-CD3 and anti-CD28 were treated with cytokine cocktail and the dNP2-ctCTLA-4 of SEQ ID NO 15 (2 μM).

K2 group: CD4 T cells activated with anti-CD3 and anti-CD28 were treated with cytokine cocktail and the K2 peptide of SEQ ID NO 1 (2 μM).

K3 group: CD4 T cells activated with anti-CD3 and anti-CD28 were treated with cytokine cocktail and the K3 peptide of SEQ ID NO 4 (2 μM).

AP-K2 group: CD4 T cells activated with anti-CD3 and anti-CD28 were treated with cytokine cocktail and the AP-K2 peptide of SEQ ID NO 9 (2 μM).

AP-K3 group: CD4 T cells activated with anti-CD3 and anti-CD28 were treated with cytokine cocktail and the AP-K3 peptide of SEQ ID NO 10 (2 μM).

### Sample Administration—Multiple Sclerosis Animal Model

The efficacy in a multiple sclerosis animal model was analyzed for six groups (PBS, dNP2-ctCTLA-4, AP, dNP2, AP-K2 and AP-K3) according to Experimental Method 2 using dNP2-ctCTLA-4, AP, dNP2, AP-K2 and AP-K3 as samples.

PBS group (control group): 100 μg of PBS was intraperitoneally injected once a day from day 7 to day 15 after the injection of PTX.

dNP2-ctCTLA-4 group (positive control group 1): The dNP2-ctCTLA-4 of SEQ ID NO 15 (100 μg) was intraperitoneally injected once a day from day 7 to day 15 after the injection of PTX.

AP group: The AP peptide of SEQ ID NO 19 (100 μg) was intraperitoneally injected once a day from day 7 to day 13 after the injection of PTX.

dNP2 group: The dNP2 peptide of SEQ ID NO 18 (100 μg) was intraperitoneally injected once a day from day 7 to day 13 after the injection of PTX.

AP-K2 group: The AP-K2 peptide of SEQ ID NO 9 (100 μg) was intraperitoneally injected once a day from day 7 to day 13 after the injection of PTX.

AP-K3 group: The AP-K3 peptide of SEQ ID NO 10 (100 μg) was intraperitoneally injected once a day from day 7 to day 13 after the injection of PTX.

After the induction of multiple sclerosis, each group was monitored every day as in Experimental Method 2 and clinical score was recorded. After the experiment was completed, lymphocytes, CD4⁺IL-17A⁺ T cells, CD4⁺IFN-γ⁺ T cells and T_{reg}/T_{eff} ratio were measured through histological analysis.

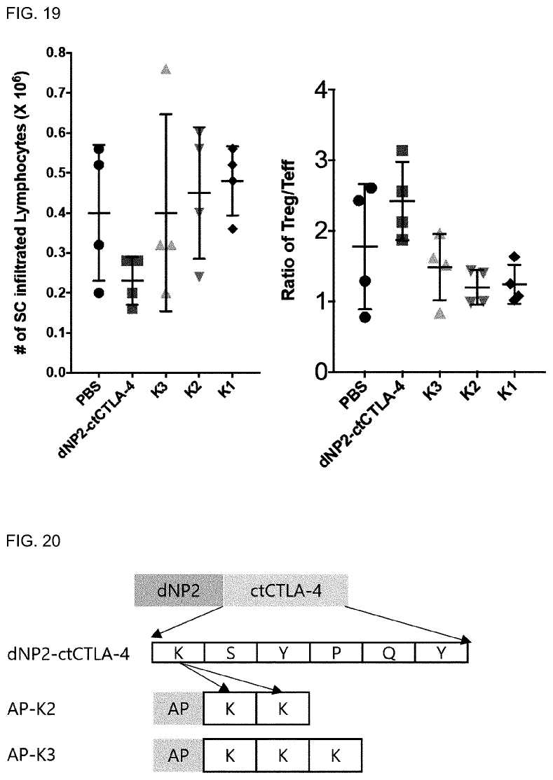

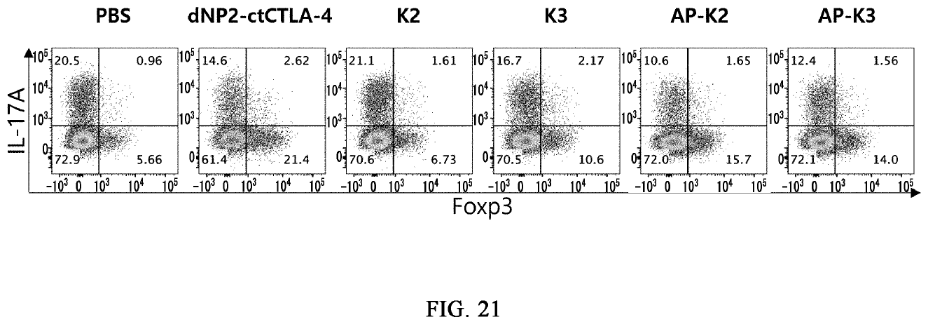

FIG. 20 shows the structure of dNP2-ctCTLA-4 (WT), a K2 peptide of SEQ ID NO 1, a K3 peptide of SEQ ID NO 4, an AP-K2 peptide of SEQ ID NO 9 and an AP-K3 peptide of SEQ ID NO 10, and FIG. 21 shows a result of flow cytometric analysis of IL-17A and foxp3 for six groups (Tₕ17 (control group), dNP2-ctCTLA-4 (WT), K2, K3, AP-K2 and AP-K3) treated to Tₕ17 cells differentiated from splenocytes isolated from mouse.

As shown in FIG. 21, the K2 group and the K3 group showed significantly better effect of suppressing immune response than the PBS (control group). The AP-K2 group and the AP-K3 group wherein the cell-penetrating peptide (CPP) such as AP was bound, showed significantly better effect of suppressing immune response than the PBS (control group). In particular, the K2 group and the K3 group, which had no CPP unlike the dNP2-ctCTLA-4 group, showed comparable effect, and the AP-K2 group and the AP-K3 group showed remarkably superior effect of inhibiting the IL-17 cytokine as compared to the dNP2-ctCTLA-4 group.

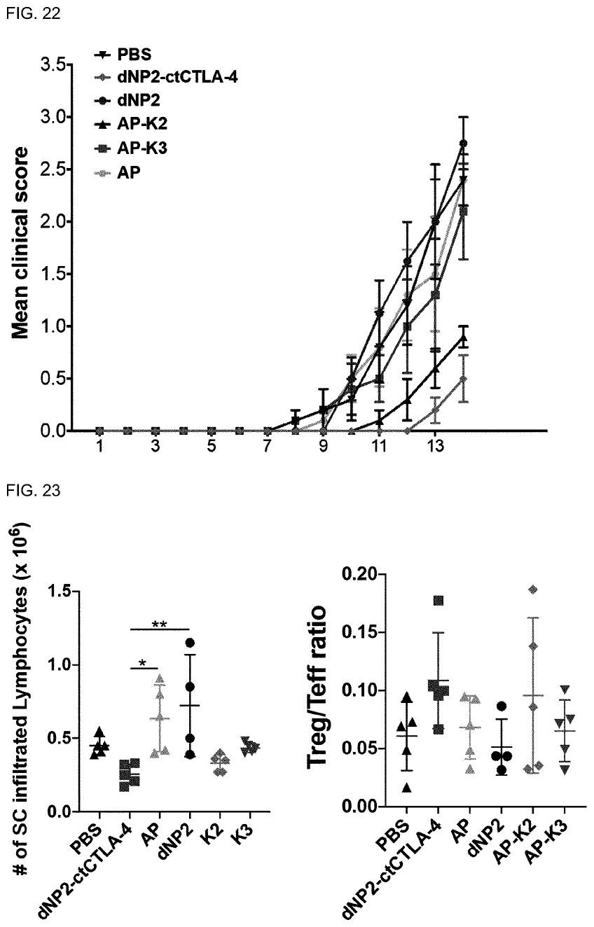

FIG. 22 shows a result of evaluating clinical symptoms of a multiple sclerosis animal model for six groups (PBS (control group), dNP2-ctCTLA-4, dNP2, AP, AP-K2 and AP-K3), and FIG. 23 shows a result of analyzing the number of infiltrated lymphocytes and immune cells (T_{reg}) for six groups (PBS (control group), dNP2-ctCTLA-4, dNP2, AP, AP-K2 and AP-K3).

As shown in FIG. 22 and FIG. 23, the AP-K2 group and the AP-K3 group showed significantly improved results for clinical symptoms as compared to the PBS (control group).

In addition, the AP-K2 group and the AP-K3 group showed significantly smaller number of infiltrated lymphocytes in the spinal cord as compared to the PBS (control group), dNP2 and AP groups.

Furthermore, the AP-K2 group and the AP-K3 group showed significantly number of regulatory T cells (T_{reg}) as compared to the PBS (control group). In particular, the AP-K2 group showed an increase comparable to that of dNP2-ctCTLA-4.

Taken together, it can be seen the K2 and K3 peptides have the prophylactic or therapeutic effect for autoimmune disease on their own, but their complexes with the cell-penetrating peptide also show superior therapeutic effect for autoimmune disease.

### Test Example 6. Analysis of Efficacy of Peptides Modified With Fatty Acids

### Sample Preparation

The degree of suppression of immune response by the K2 peptide and the K3 peptide modified with a fatty acid was analyzed.

First, dNP2-ctCTLA-4, K2, K3, Myr-K2 and Myr-K3 were designed as described in Table 8 and were synthesized according to Examples 1, 4, 15 and 16. Myr-K2 and Myr-K3 are peptides modified with a fatty acid, prepared in Examples 15 and 16. Myr indicates myristic acid.

TABLE 8

| SEQ | | |
|---|---|---|
| ID | | |
| NO | Name | Amino acid sequence |
| 15 | dNP2- | KIKKVKKKGRKGSKIKKVKKKGRKGGKMLKKRSP |
| | ctCTLA-4 | LTTGVYVKMPPTEPECEKQFQPYFIPIN |
| 1 | K2 | KMLKKRSKMLKKRS |
| 4 | K3 | KMLKKRSKMLKKRSKMLKKRS |
| 9 | AP-K2 | RRRWCKRRRGGKMLKKRSKMLKKRS |

### Sample Administration—Tₕ17 Differentiation Condition

The efficacy under Tₕ17 differentiation condition was analyzed in the same manner as in Experimental Method 1 except that dNP2-ctCTLA-4, K2, K3, Myr-K2 and Myr-K3 were used as samples. Flow cytometry was conducted for six groups (PBS, dNP2-ctCTLA-4, K2, K3, Myr-K2 and Myr-K3) using IL-17 and Foxp3 fluorescent antibodies 4 days later.

Tₕ17 group (control group): CD4 T cells activated with anti-CD3 and anti-CD28 were treated with cytokine cocktail and PBS (2 μM).

dNP2-ctCTLA-4 group (positive control group): CD4 T cells activated with anti-CD3 and anti-CD28 were treated with cytokine cocktail and the (dNP2-ctCTLA-4)_WT of SEQ ID NO 15 (2 μM).

K2 group: CD4 T cells activated with anti-CD3 and anti-CD28 were treated with cytokine cocktail and the K2 peptide of SEQ ID NO 1 (2 μM).

K3 group: CD4 T cells activated with anti-CD3 and anti-CD28 were treated with cytokine cocktail and the K3 peptide of SEQ ID NO 4 (2 μM).

Myr-K2 group: CD4 T cells activated with anti-CD3 and anti-CD28 were treated with cytokine cocktail and the Myr-K2 of Example 15 (2 μM).

Myr-K3 group: CD4 T cells activated with anti-CD3 and anti-CD28 were treated with cytokine cocktail and the Myr-K3 of Example 16 (2 μM).

### Sample Administration—Multiple Sclerosis Animal Model

The efficacy in a multiple sclerosis animal model was analyzed according to Experimental Method 2 for six groups (PBS, dNP2-ctCTLA-4, K2, AP-K2, Myr-K2 and Myr-K3) using dNP2-ctCTLA-4, K2, AP-K2, Myr-K2 and Myr-K3 as samples.

PBS group (control group): 100 μg of PBS was intraperitoneally injected once a day from day 7 to day 13 after the injection of PTX.

dNP2-ctCTLA-4 group (positive control group): The (dNP2-ctCTLA-4)_WT of SEQ ID NO 15(100 μg) was intraperitoneally injected once a day from day 7 to day 13 after the injection of PTX.

K2 group: The K2 peptide of SEQ ID NO 1 (100 μg) was intraperitoneally injected once a day from day 7 to day 13 after the injection of PTX.

AP-K2 group: The AP-K2 peptide of SEQ ID NO 9 (100 μg) was intraperitoneally injected once a day from day 7 to day 13 after the injection of PTX.

Myr-K2 group: The Myr-K2 of Example 15 (100 μg) was intraperitoneally injected once a day from day 7 to day 13 after the injection of PTX.

Myr-K3 group: The Myr-K3 of Example 16 (100 μg) was intraperitoneally injected once a day from day 7 to day 13 after the injection of PTX.

After the induction of multiple sclerosis, each group was monitored every day as in Experimental Method 2 and clinical score was recorded. After the experiment was completed, lymphocytes, CD4⁺IL-17A⁺ T cells, CD4⁺IFN-γ⁺ T cells and T_{reg}/T_{eff} ratio may be measured through histological analysis.

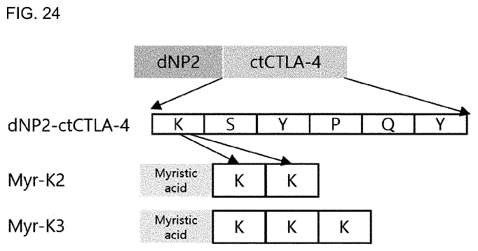

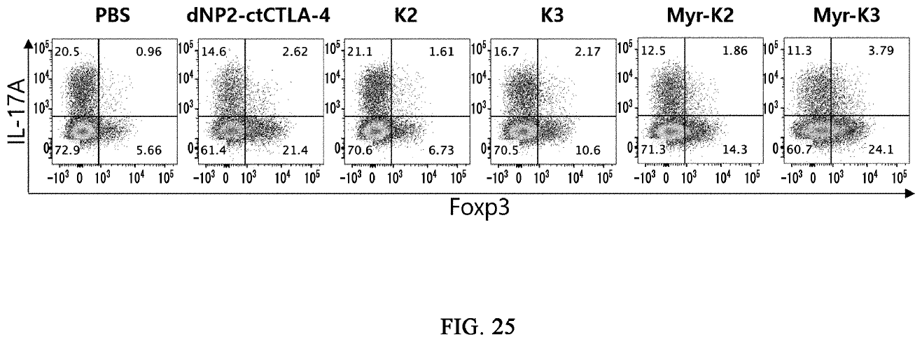

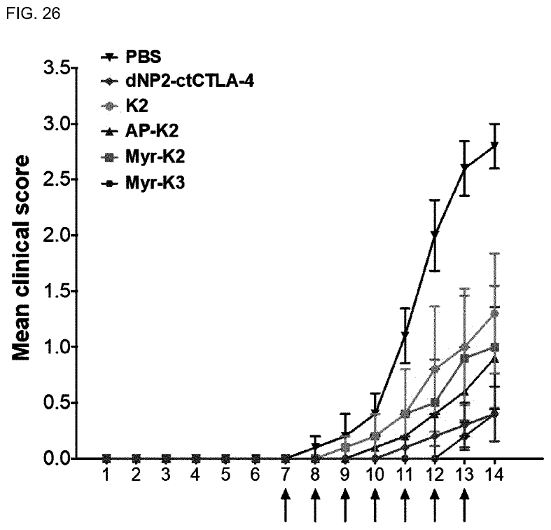

FIG. 24 shows the structure of dNP2-ctCTLA-4 (WT), a K2 peptide of SEQ ID NO 1, a K3 peptide of SEQ ID NO 4, an AP-K2 peptide of Example 15 and an AP-K3 peptide of Example 16, and FIG. 25 shows a result of flow cytometric analysis of IL-17A and foxp3 for six groups (Tₕ17(control group), dNP2-ctCTLA-4, K2, K3, Myr-K2 and Myr-K3) treated to Tₕ17 cells differentiated from splenocytes isolated from mouse.

As shown in FIG. 25, the K2 group and the K3 group showed significantly superior effect of suppressing immune response as compared to the PBS group. The Myr-K2 and the Myr-K3 wherein the fatty acid was bound to the K2 peptide and the K3 peptide also showed significantly superior effect of suppressing immune response as compared to the PBS group. In particular, the Myr-K3 showed remarkably significantly superior effect of suppressing immune response as compared to the dNP2-ctCTLA-4 group.

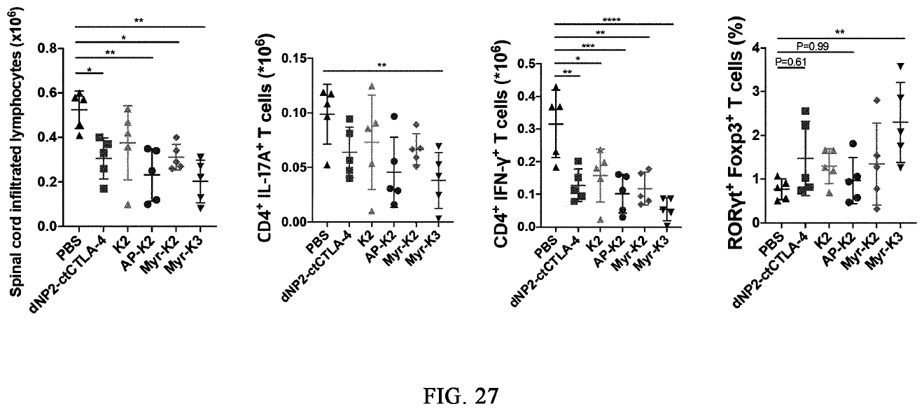

FIG. 26 shows a result of evaluating clinical symptoms of a multiple sclerosis animal model for six groups (Tₕ17, dNP2-ctCTLA-4, K2, AP-K2, Myr-K2 and Myr-K3), and FIG. 27 shows a result of analyzing the number of infiltrated lymphocytes and immune cells (T_{reg}) for six groups (Tₕ17, dNP2-ctCTLA-4, K2, AP-K2, Myr-K2 and Myr-K3).

As shown in FIG. 26 and FIG. 27, not only the K2 group and the AP-K2 group but also the Myr-K2 and Myr-K3 groups showed significantly improved result for clinical symptoms as compared to the PBS group. In particular, the Myr-K3 group showed significant improvement comparable to that of the dNP2-ctCTLA-4 group.

In addition, not only the K2 group and the AP-K2 group but also the Myr-K2 and Myr-K3 groups showed significantly smaller number of infiltrated lymphocytes in the spinal cord than the PBS (control group). The AP-K2 group and the Myr-K3 group showed the most superior effects. In particular, the Myr-K3 group showed a smaller number of infiltrated lymphocytes than the dNP2-ctCTLA-4 group.

In addition, not only the AP-K2 group but also the Myr-K2 and Myr-K3 groups showed inhibited expression of II-17 T cells and IFN-γ T cells and increase of Foxp3 T cells as compared to the PBS (control group). In particular, the Myr-K3 group showed better inhibition than the dNP2-ctCTLA-4 group.

Taken together, it can be seen that not only the K2 and K3 peptides but also Myr-K2 and Myr-K3 have superior prophylactic and therapeutic effects for autoimmune disease.

### Test Example 7. Analysis of Efficacy for Regulatory T Cell-Inducing Peptides Having D-Amino Acids

### Sample Preparation

Experiment was conducted to reconfirm the effect of the regulatory T cell-inducing peptide of SEQ ID NO 1 or SEQ ID NO 4 wherein some amino acid residues are replaced with D-amino acid residues for autoimmune disease. First, dNP2-ctCTLA-4, Myr-K3, Myr-K3D, Pal-K3, Myr-Ap-K2D and Pal-Ap-K2D were prepared. Specifically, dNP2-ctCTLA-4 was prepared in the same manner as in Test Example 1 and Myr-K3, Myr-K3D, Pal-K3, Myr-Ap-K2D and Pal-Ap-K2D were prepared according to Example 16, Example 17, Example 18, Example 19 and Example 20.

### Sample Administration—Tₕ17 Differentiation Condition

The efficacy under Tₕ17 differentiation conditions was analyzed in the same manner as in Experimental Method 1 except that dNP2-ctCTLA-4, Myr-K3, Myr-K3D, Pal-K3, Myr-Ap-K2D and Pal-Ap-K2D were used as samples. Flow cytometry was conducted for seven groups (Tₕ17, dNP2-ctCTLA-4, Myr-K3, Myr-K3D, Pal-K3, Myr-Ap-K2D and Pal-Ap-K2D) using IL-17 and Foxp3 fluorescent antibodies 4 days later.

Tₕ17 group (control group 1): CD4 T cells activated with anti-CD3 and anti-CD28 were treated with cytokine cocktail and PBS (2 μM).

dNP2-ctCTLA-4 group (positive control group): CD4 T cells activated with anti-CD3 and anti-CD28 were treated with cytokine cocktail and the (dNP2-ctCTLA-4)_WT of SEQ ID NO 15 (2 μM).

Myr-K3 group: CD4 T cells activated with anti-CD3 and anti-CD28 were treated with cytokine cocktail and the Myr-K3 of Example 16 (2 μM).

Myr-K3D group: CD4 T cells activated with anti-CD3 and anti-CD28 were treated with cytokine cocktail and the Myr-K3D of Example 17 (2 μM).

Pal-K3 group: CD4 T cells activated with anti-CD3 and anti-CD28 were treated with cytokine cocktail and the Pal-K3 of Example 18 (2 μM).

Myr-AP-K2D group: CD4 T cells activated with anti-CD3 and anti-CD28 were treated with cytokine cocktail and the Myr-AP-K2D of Example 19 (2 μM).

Pal-AP-K2D group: CD4 T cells activated with anti-CD3 and anti-CD28 were treated with cytokine cocktail and the Pal-AP-K2D (2 μM) of Example 20.

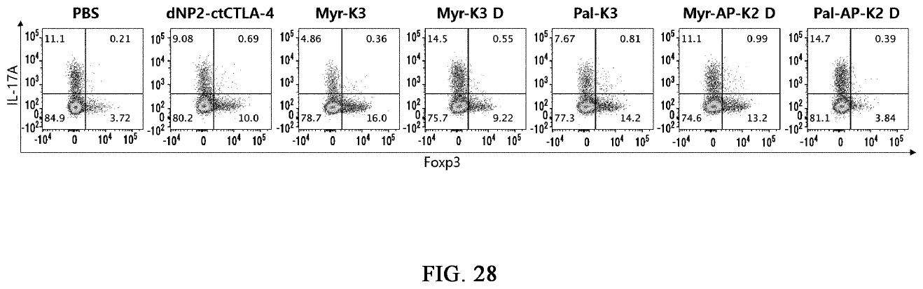

FIG. 28 shows a result of flow cytometric analysis of IL-17A and foxp3 for seven groups (Tₕ17, dNP2-ctCTLA-4, Myr-K3, Myr-K3D, Pal-K3, Myr-Ap-K2D and Pal-Ap-K2D) treated to Tₕ17 cells differentiated from splenocytes isolated from mouse.

As shown in FIG. 28, the expression of T_{reg} cells was significantly increased in the Myr-K3D group, Myr-Ap-K2D group and the Pal-AP-K2D group having D-amino acid residues as compared to the PBS group. In particular, the K3 peptide showed remarkably superior effect of suppressing immune response for both myristic acid (Myr) and palmitic acid (Pal). Among the fatty acids, palmitic acid is advantageous in terms of large-scale production. It was confirmed that superior efficacy was maintained even when palmitic acid was bound to the peptide.

For the K2 peptide and the K3 peptide substituted with D-amino acid residues, stability was improved and the ability of regulating immune function when excessive immune response was induced by T_{reg} cells was significantly improved as compared to L-amino acid residues.

### Test Example 8. Analysis of Efficacy for K2 Variants

### Sample Preparation

In order to investigate the role of amino acid residues constituting the AP-K2 peptide, variants with some amino acid residues of the AP-K2 peptide of SEQ ID NO 9 replaced with alanine were designed as SEQ ID NOS 42-48 and they were prepared by AnyGen (Kwangju, Korea) using solid-phase peptide synthesis (SPPS). The specific preparation process is the same as in Example 1 and the prepared peptides also showed purity of 95% or higher.

In SEQ ID NO 42, the lysine and arginine residues which are expected to play major roles in the AP-K2 peptide were replaced with alanine (A) as a negative control group (negative control) and the AP peptide represented by SEQ ID NO 19 was used as a comparison group (The AP-K2 KA used in this test example is different from the dNP2-KA of SEQ ID NO 19).

SEQ ID NO 9: AP-K2
RRRWCKRRRGGKMLKKRSKMLKKRS
SEQ ID NO 19: AP-K2
RRRWCKRRR
SEQ ID NO 42: AP-K2 KA
RRRWCKRRRGGAMLAAASAMLAAAS
SEQ ID NO 43: AP-K2 1KA
RRRWCKRRRGGAMLKKRSAMLKKRS
SEQ ID NO 44: AP-K2 MA
RRRWCKRRRGGKALKKRSKALKKRS
SEQ ID NO 45: AP-K2 LA
RRRWCKRRRGGKMAKKRSKMAKKRS
SEQ ID NO 46: AP-K2 2KA
RRRWCKRRRGGKMLAKRSKMLAKRS
SEQ ID NO 47: AP-K2 3KA
RRRWCKRRRGGKMLKARSKMLKARS
SEQ ID NO 48: AP-K2 RA
RRRWCKRRRGGKMLKKASKMLKKAS
SEQ ID NO 49: AP-K2 SA
RRRWCKRRRGGKMLKKRAKMLKKRA

### Sample Administration—Differentiation of Tₕ17

The efficacy under Tₕ17 differentiation conditions was analyzed in the same manner as in Experimental Method 1 except that AP-K2 and AP-K2 variants (KA, IKA, MA, LA, 2KA, 3KA, RA and SA) were used as samples. Flow cytometry was conducted for each group using IL-17 and Foxp3 fluorescent antibodies 4 days later.

Tₕ17 group (PBS; control group): CD4 T cells activated with anti-CD3 and anti-CD28 were treated with cytokine cocktail and PBS of the same volume as a positive control group.

AP-K2 group (positive control group): CD4 T cells activated with anti-CD3 and anti-CD28 were treated with cytokine cocktail and the AP-K2 of SEQ ID NO 9 (2 μM).

AP group (AP; comparison group): CD4 T cells activated with anti-CD3 and anti-CD28 were treated with cytokine cocktail and the AP of SEQ ID NO 19 (2 μM).

AP-K2 KA group: CD4 T cells activated with anti-CD3 and anti-CD28 were treated with cytokine cocktail and the AP-K2 KA of SEQ ID NO 41 (2 μM).

AP-K2 IKA group: CD4 T cells activated with anti-CD3 and anti-CD28 were treated with cytokine cocktail and the AP-K2 IKA of SEQ ID NO 42 (2 μM).

AP-K2 MA group: CD4 T cells activated with anti-CD3 and anti-CD28 were treated with cytokine cocktail and the AP-K2 MA of SEQ ID NO 43 (2 μM).

AP-K2 LA group: CD4 T cells activated with anti-CD3 and anti-CD28 were treated with cytokine cocktail and the AP-K2 LA of SEQ ID NO 44 (2 μM).

AP-K2 2KA group: CD4 T cells activated with anti-CD3 and anti-CD28 were treated with cytokine cocktail and the AP-K2 2KA of SEQ ID NO 45 (2 μM).

AP-K2 3KA group: CD4 T cells activated with anti-CD3 and anti-CD28 were treated with cytokine cocktail and the AP-K2 3KA of SEQ ID NO 46 (2 μM).

AP-K2 RA group: CD4 T cells activated with anti-CD3 and anti-CD28 were treated with cytokine cocktail and the AP-K2 RA of SEQ ID NO 47 (2 μM).

AP-K2 SA group: CD4 T cells activated with anti-CD3 and anti-CD28 were treated with cytokine cocktail and the AP-K2 SA of SEQ ID NO 48 (2 μM).

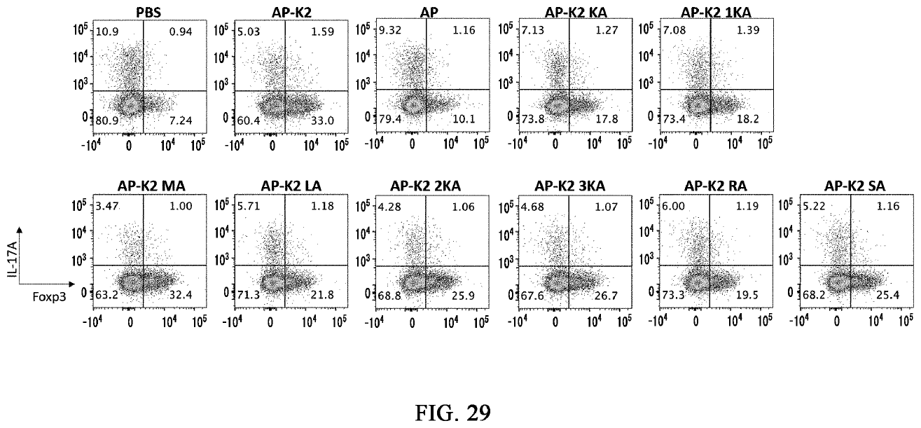

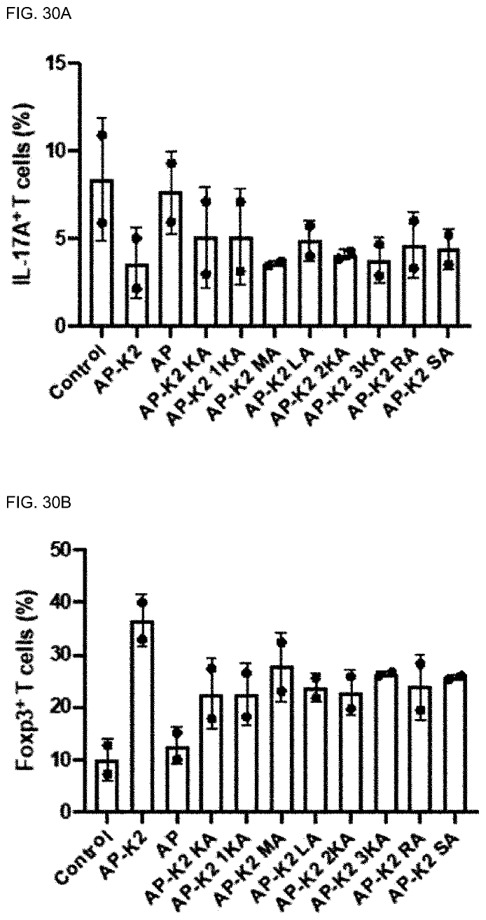

FIG. 29 shows a result of flow cytometric analysis of IL-17A and foxp3 for eleven groups (Tₕ17, AP-K2, AP, AP-K2 KA, AP-K2 IKA, AP-K2 MA, AP-K2 LA, AP-K2 2KA, AP-K2 3KA, AP-K2 RA and AP-K2 SA) treated to Tₕ17 cells differentiated from splenocytes isolated from mouse, and FIGS. 30A and 30B show a result of flow cytometric analysis of CD4⁺IL-17A⁺ T cells (FIG. 30A) and CD4⁺foxp3⁺ T cells (FIG. 30B) for eleven groups (Tₕ17, AP-K2, AP, AP-K2 KA, AP-K2 IKA, AP-K2 MA, AP-K2 LA, AP-K2 2KA, AP-K2 3KA, AP-K2 RA and AP-K2 SA).

In order to more accurately analyze the amino acid residues that play a major role in the K2 motif, the effect of suppressing immune response was investigated for the variants substituted with different amino acid residues.

As shown in FIGS. 29 and 30, the AP-K2 group increased Foxp3 by about 5 times or more and reduced IL-17 to half or less as compared to the control group. Through this, it can be seen that the AP-K2 peptide (SEQ ID NO 9) of the present disclosure exhibits superior effect of suppressing immune response.

In contrast, the function of AP-K2 was decreased slightly in the AP-K2 KA group and was decreased significantly in the AP-K2 1KA and AP-K2 RA groups. Through this, it can be seen that the first lysine residue and arginine residue in the AP-K2 play major roles in the suppression of immune response.

The slightly decreased function of AP-L2 in the AP-K2 LA group indicates that the leucine residue in the AP-K2 also plays a major role.

In addition, since the reduction of IL-17 and the increase of Foxp3 were in the order of the AP-K2 SA group, the AP-K2 2KA group, the AP-K2 3KA group and the AP-K2 MA group, it can be seen that the serine residue, the second and third lysine residues and the methionine residue in the K2 motif are involved in the suppression of immune response in that order.

While specific exemplary embodiments of the present disclosure have been described in detail, it will be obvious to those having ordinary knowledge in the art that they are merely specific exemplary embodiments and the scope of the present disclosure is not limited by them. It is to be understood that the substantial scope of the present disclosure is defined by the appended claims and their equivalents.

1. A regulatory T cell-inducing peptide represented by the following sequence:
[Lys-Xaa1-Leu-Xaa2-Xaa3-Arg-Xaa4]ₙ
wherein
each of Xaa1, Xaa2, Xaa3 and Xaa4 is independently an amino acid selected from alanine (A), valine (V), isoleucine (I), leucine (L), methionine (M), phenylalanine (F), tyrosine (Y), tryptophan (W), lysine (K), arginine (R), histidine (H), serine (S), threonine (T), asparagine (N), glutamine (Q), aspartate (D), glutamate (E), cysteine (C), glycine (G) and proline (P),
n is an integer 2 or 3, and
one or more amino acid residue selected from the sequence is an L- or D-amino acid residue.

2. The regulatory T cell-inducing peptide according to claim 1, wherein, in the sequence, Xaa1 is a hydrophobic amino acid selected from alanine (A), valine (V), isoleucine (I), leucine (L), methionine (M), phenylalanine (F), tyrosine (Y) and tryptophan (W).

3. The regulatory T cell-inducing peptide according to claim 2, wherein, in the sequence, each of Xaa2 and Xaa3 is independently a basic amino acid selected from lysine (K), arginine (R) and histidine (H).

4. The regulatory T cell-inducing peptide according to claim 3, wherein, in the sequence, Xaa4 is a polar amino acid selected from serine (S), threonine (T), asparagine (N) and glutamine (Q).

5. The regulatory T cell-inducing peptide according to claim 1, wherein, in the sequence, Xaa1 is methionine (M).

6. The regulatory T cell-inducing peptide according to claim 5, wherein, in the sequence, Xaa2 and Xaa3 are lysine (K).

7. The regulatory T cell-inducing peptide according to claim 6, wherein, in the sequence, Xaa4 is serine (S).

8. The regulatory T cell-inducing peptide according to claim 1, wherein, in the sequence, one or more amino acid residue selected from the first, second, sixth and seventh positions is a D-amino acid.

9. The regulatory T cell-inducing peptide according to claim 1, wherein, in the sequence, the amino acid residues at the sixth and seventh positions are D-amino acids.

10. The regulatory T cell-inducing peptide according to claim 1, wherein the regulatory T cell-inducing peptide further comprises a linker.

11. The regulatory T cell-inducing peptide according to claim 10, wherein the linker is selected from a group consisting of (GGGGS)ₘ (1≤m≤4), (GG)ₘ (1≤m≤4), (GSSGGS)ₘ (1≤m≤4), (EAAAK)ₘ (1≤m≤4), PAPAP, (AP)ₘ (1≤m≤4), A(EAAAK)ₘ (1≤m≤4), (RR)ₘ (1≤m≤4) and GFLG.

12. The regulatory T cell-inducing peptide according to claim 1, wherein a fatty acid is additionally bound to the N- or C-terminal of the regulatory T cell-inducing peptide.

13. The regulatory T cell-inducing peptide according to claim 12, wherein the fatty acid is bound to the N-terminal of the regulatory T cell-inducing peptide via an amide bond.

14. The regulatory T cell-inducing peptide according to claim 12, wherein the fatty acid is a C₁₀₋₂₀ saturated or unsaturated fatty acid.

15. The regulatory T cell-inducing peptide according to claim 12, wherein the fatty acid is one or more selected from a group consisting of myristic acid, stearic acid, linoleic acid, palmitic acid, oleic acid and lauric acid.

16. The regulatory T cell-inducing peptide according to claim 1, wherein a cell-penetrating peptide (CPP) is additionally bound to the N- or C-terminal of the regulatory T cell-inducing peptide.

17. The regulatory T cell-inducing peptide according to claim 16, wherein the cell-penetrating peptide is one or more selected from a group consisting of dNP2 represented by SEQ ID NO 18, AP represented by SEQ ID NO 19, TAT (HIV 1 trans-activating protein), a polyarginine polypeptide having 6 to 8 arginines, a polylysine polypeptide having 7 to 11 lysines and iRGD (internalizing RGD).

18. The regulatory T cell-inducing peptide according to claim 1, wherein the regulatory T cell-inducing peptide is any one selected from a group consisting of SEQ ID NOS 1-6, SEQ ID NO 11, SEQ ID NO 12 and SEQ ID NOS 50-54.

19. The regulatory T cell-inducing peptide according to claim 16, wherein the regulatory T cell-inducing peptide with the cell-penetrating peptide bound is any one selected from a group consisting of SEQ ID NOS 7-10, SEQ ID NO 13 and SEQ ID NO 14.

20. A pharmaceutical composition for preventing or treating autoimmune disease, comprising the regulatory T cell-inducing peptide according to claim 1 as an active ingredient.

21. The pharmaceutical composition for preventing or treating autoimmune disease according to claim 20, wherein the autoimmune disease is one or more selected from a group consisting of lupus (systemic lupus erythematosus), rheumatoid arthritis, Crohn's disease, ulcerative colitis, systemic scleroderma (progressive systemic sclerosis), atopic dermatitis, psoriasis, pemphigus, asthma, aphthous stomatitis, chronic thyroiditis, inflammatory enteritis, multiple sclerosis, mixed connective tissue disease, autoimmune hemolytic anemia, Behçet's disease, autoimmune encephalomyelitis, myasthenia gravis, autoimmune thyroiditis, polyarteritis nodosa, ankylosing spondylitis, fibromyalgia syndrome, Sjögren syndrome, autoimmune uveitis, chronic inflammatory demyelinating polyneuropathy and temporal arteritis.

22. The pharmaceutical composition for preventing or treating autoimmune disease according to claim 20, wherein the composition inhibits the activity of Tₕ17 and activates T_{reg}.


SEQUENCE LISTING (54 sequences)

SEQ ID NO: 1 - protein, 14 aa, Artificial Sequence
  K2
KMLKKRSKMLKKRS

SEQ ID NO: 2 - protein, 19 aa, Artificial Sequence
  K2(G4S)
KMLKKRSGGGGSKMLKKRS

SEQ ID NO: 3 - protein, 16 aa, Artificial Sequence
  K2(GG)
KMLKKRSGGKMLKKRS

SEQ ID NO: 4 - protein, 21 aa, Artificial Sequence
  K3
KMLKKRSKMLKKRSKMLKKRS

SEQ ID NO: 5 - protein, 31 aa, Artificial Sequence
  K3(G4S)
KMLKKRSGGGGSKMLKKRSGGGGSKMLKKRS

SEQ ID NO: 6 - protein, 25 aa, Artificial Sequence
  K3(GG)
KMLKKRSGGKMLKKRSGGKMLKKRS

SEQ ID NO: 7 - protein, 38 aa, Artificial Sequence
  dNP2-K2
KIKKVKKKGRKGSKIKKVKKKGRKKMLKKRSKMLKKRS

SEQ ID NO: 8 - protein, 45 aa, Artificial Sequence
  dNP2-K3
KIKKVKKKGRKGSKIKKVKKKGRKKMLKKRSKMLKKRSKMLKKRS

SEQ ID NO: 9 - protein, 25 aa, Artificial Sequence
  AP-K2
RRRWCKRRRGGKMLKKRSKMLKKRS

SEQ ID NO: 10 - protein, 32 aa, Artificial Sequence
  AP-K3
RRRWCKRRRGGKMLKKRSKMLKKRSKMLKKRS

SEQ ID NO: 11 - protein, 14 aa, Artificial Sequence
  KD2  The 1st, 2nd, 5th, 6th, 12th and 13th amino acid residues are D-form amino acids.
KMLKKRSKMLKKRS

SEQ ID NO: 12 - protein, 21 aa, Artificial Sequence
  KD3  The 1st, 2nd, 5th, 6th, 12th, 13th, 19th and 20th amino acid residues are D-form amino acids.
KMLKKRSKMLKKRSKMLKKRS

SEQ ID NO: 13 - protein, 25 aa, Artificial Sequence
  AP-KD2  The 1st, 2nd, 17th, 18th, 24th and 25th amino acid residues are D-form amino acids.
RRRWCKRRRGGKMLKKRSKMLKKRS

SEQ ID NO: 14 - protein, 32 aa, Artificial Sequence; identical to SEQ ID NO: 10
  AP-KD3  The 1st, 2nd, 17th, 18th, 24th, 25th, 31st and 32nd amino acid residues are D-form amino acids.

SEQ ID NO: 15 - protein, 62 aa, Artificial Sequence
  dNP2-ctCTLA-4
KIKKVKKKGRKGSKIKKVKKKGRKGGKMLKKRSPLTTGVYVKMPPTEPECEKQFQPYFIPIN

SEQ ID NO: 16 - protein, 63 aa, Artificial Sequence
  TAT-ctCTLA-4
YGRKKRRRQRRRGSKIKKVKKKGRKGGKMLKKRSPLTTGVYVKMPPTEPECEKQFQPYFIPIN

SEQ ID NO: 17 - protein, 47 aa, Artificial Sequence
  AP-ctCTLA-4
RRRWCKRRRGGKMLKKRSPLTTGVYVKMPPTEPECEKQFQPYFIPIN

SEQ ID NO: 18 - protein, 24 aa, Artificial Sequence
  dNP2
KIKKVKKKGRKGSKIKKVKKKGRK

SEQ ID NO: 19 - protein, 9 aa, Artificial Sequence
  AP
RRRWCKRRR

SEQ ID NO: 20 - protein, 62 aa, Artificial Sequence
  dNP2-KA
KIKKVKKKGRKGSKIKKVKKKGRKGGAMLAAASPLTTGVYVKMPPTEPECEKQFQPYFIPIN

SEQ ID NO: 21 - protein, 62 aa, Artificial Sequence
  dNP2-PA
KIKKVKKKGRKGSKIKKVKKKGRKGGKMLKKRSPLTTGVYVKMAATEAECEKQFQPYFIPIN

SEQ ID NO: 22 - protein, 62 aa, Artificial Sequence
  dNP2-YF
KIKKVKKKGRKGSKIKKVKKKGRKGGKMLKKRSPLTTGVFVKMPPTEPECEKQFQPFFIPIN

SEQ ID NO: 23 - protein, 56 aa, Artificial Sequence
  dNP2-KSYPQ
KIKKVKKKGRKGSKIKKVKKKGRKGGKMLKKRSPLTTGVYVKMPPTEPECEKQFQP

SEQ ID NO: 24 - protein, 51 aa, Artificial Sequence
  dNP2-KSYP
KIKKVKKKGRKGSKIKKVKKKGRKGGKMLKKRSPLTTGVYVKMPPTEPECE

SEQ ID NO: 25 - protein, 43 aa, Artificial Sequence
  dNP2-KSY
KIKKVKKKGRKGSKIKKVKKKGRKGGKMLKKRSPLTTGVYVKM

SEQ ID NO: 26 - protein, 39 aa, Artificial Sequence
  dNP2-KS
KIKKVKKKGRKGSKIKKVKKKGRKGGKMLKKRSPLTTGV

SEQ ID NO: 27 - protein, 31 aa, Artificial Sequence
  dNP2-K1
KIKKVKKKGRKGSKIKKVKKKGRKKMLKKRS

SEQ ID NO: 28 - protein, 7 aa, Artificial Sequence
  K1
KMLKKRS

SEQ ID NO: 29 - protein, 7 aa, Artificial Sequence
  S
SPLTTGV

SEQ ID NO: 30 - protein, 4 aa, Artificial Sequence
  Y1
YVKM

SEQ ID NO: 31 - protein, 8 aa, Artificial Sequence
  P
PPTEPECE

SEQ ID NO: 32 - protein, 6 aa, Artificial Sequence
  Y2
YFIPIN

SEQ ID NO: 33 - protein, 6 aa, Artificial Sequence
  R6
RRRRRR

SEQ ID NO: 34 - protein, 7 aa, Artificial Sequence
  R7
RRRRRRR

SEQ ID NO: 35 - protein, 8 aa, Artificial Sequence
  R8
RRRRRRRR

SEQ ID NO: 36 - protein, 9 aa, Artificial Sequence
  iRGD
CRGDKGPDC

SEQ ID NO: 37 - protein, 7 aa, Artificial Sequence
  L7
LLLLLLL

SEQ ID NO: 38 - protein, 8 aa, Artificial Sequence
  L8
LLLLLLLL

SEQ ID NO: 39 - protein, 9 aa, Artificial Sequence
  L9
LLLLLLLLL

SEQ ID NO: 40 - protein, 10 aa, Artificial Sequence
  L10
LLLLLLLLLL

SEQ ID NO: 41 - protein, 11 aa, Artificial Sequence
  L11
LLLLLLLLLLL

SEQ ID NO: 42 - protein, 25 aa, Artificial Sequence
  AP-K2 KA
RRRWCKRRRGGAMLAAASAMLAAAS

SEQ ID NO: 43 - protein, 25 aa, Artificial Sequence
  AP-K2 1KA
RRRWCKRRRGGAMLKKRSAMLKKRS

SEQ ID NO: 44 - protein, 25 aa, Artificial Sequence
  AP-K2 MA
RRRWCKRRRGGKALKKRSKALKKRS

SEQ ID NO: 45 - protein, 25 aa, Artificial Sequence
  AP-K2 LA
RRRWCKRRRGGKMAKKRSKMAKKRS

SEQ ID NO: 46 - protein, 25 aa, Artificial Sequence
  AP-K2 2KA
RRRWCKRRRGGKMLAKRSKMLAKRS

SEQ ID NO: 47 - protein, 25 aa, Artificial Sequence
  AP-K2 3KA
RRRWCKRRRGGKMLKARSKMLKARS

SEQ ID NO: 48 - protein, 25 aa, Artificial Sequence
  AP-K2 RA
RRRWCKRRRGGKMLKKASKMLKKAS

SEQ ID NO: 49 - protein, 25 aa, Artificial Sequence
  AP-K2 SA
RRRWCKRRRGGKMLKKRAKMLKKRA

SEQ ID NO: 50 - protein, 14 aa, Artificial Sequence
  AP-K2 MA
KALKKRSKALKKRS

SEQ ID NO: 51 - protein, 14 aa, Artificial Sequence
  AP-K2 LA
KMAKKRSKMAKKRS

SEQ ID NO: 52 - protein, 14 aa, Artificial Sequence
  AP-K2 2KA
KMLAKRSKMLAKRS

SEQ ID NO: 53 - protein, 14 aa, Artificial Sequence
  AP-K2 3KA
KMLKARSKMLKARS

SEQ ID NO: 54 - protein, 14 aa, Artificial Sequence
  AP-K2 SA
KMLKKRAKMLKKRA